(19) United States
(12) Patent Application Publication
(10) Pub. No.: US 2011/0160563 A1
(43) Pub. Date: Jun. 30, 2011

(54) DIAGNOSTIC SKIN MAPPING BY MRS, MRI AND OTHER METHODS

(76) Inventors: Richard G. Glogau, San Francisco, CA (US); Bernhard P.J. Blümich, Rott (DE); Alexander Pines, Berkeley, CA (US)

(21) Appl. No.: 12/919,183

(22) PCT Filed: Feb. 26, 2009
(86) PCT No.: PCT/US09/35308

§ 371 Date: Feb. 25, 2011

Related U.S. Application Data
(60) Provisional application No. 61/031,604, filed on Feb. 26, 2008.

(51) Int. Cl. A61B 5/055

(52) U.S. Cl. 600/410

(57) ABSTRACT

This invention pertains to improved methods of classifying skin types as well as improved methods for determining the appropriateness of products and evaluating methods for treating particular skin. The methods typically utilize a “skin type” database containing one or more quantitative measures (e.g., NMR data) of skin properties. The database can optionally include various qualitative measures of skin as well (e.g., Glogau scale and/or Fitzpatrick scale values).

### CROSS-REFERENCE TO RELATED APPLICATIONS

This application claims priority to and benefit of U.S. Ser. No. 61/031,604, filed on Feb. 26, 2008, which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with government support under Contract No. DE-AC02-05CH11231 awarded by the U.S. Department of Energy. The government has certain rights in the invention.

### FIELD OF THE INVENTION

This invention pertains to the field of dermatology and reconstructive or cosmetic surgery. In particular this invention pertains to the development of a novel objective measure of characterizing skin types. The methods find use in treatment planning, cosmetology, and research in dermatological methods and therapeutics.

### BACKGROUND OF THE INVENTION

There are two skin type classification systems that are currently used in the dermatology field. The first is based on the skin's reactivity to the sun and was developed by Fitzpatrick in 1963 (Fitzpatrick et al. (1963) Dermatol Wochenschr 147: 481-489). This grading scale is used universally but only takes into account the skin's pigmentation and reaction to sun exposure. More recently, a scale was developed to rank the degree of photodamage or skin aging caused by the sun. This “Glogau Photoaging scale” divides skin into four types according to the amount of wrinkles that are present (see, e.g., Glogau (1996) Semin Cutan Med Surg, 15(3): 134-138). There are no widely accepted skin typing systems that take into account wrinkles, pigmentation, dryness and sensitivity. In addition, there are no widely used systems to type hair. These systems, however, are rather qualitative, and results can vary depending on the person making the evaluation.

### SUMMARY

This invention pertains to improved methods of classifying skin types as well as improved methods for determining the appropriateness of products and evaluating methods for treating particular skin. The methods typically involve one or more quantitative measurements (e.g., NMR, MRI, PET, etc.) of the skin at one or more regions in a subject of interest. In certain embodiments the quantitative measurements can be used directly to characterize the measured skin and/or they can be used in conjunction with a “skin type” database containing one or more quantitative measures (e.g., NMR data) of skin properties and/or parameters calculated therefrom to characterize the skin. The dataset can optionally include various qualitative measures of skin as well. In various methods of characterizing skin types using quantitative measurements (e.g., NMR), skin type databases, methods of use of such, treatment methods involving quantitative measures of skin and/or skin-type databases, and the like are provided herein.

Accordingly, in certain embodiments, methods are provided of characterizing skin type in a subject (e.g., a human subject). The methods typically involve nuclear magnetic resonance (NMR) measurements at a plurality of skin depths at one or more locations on the subject; and calculating from data provided by the NMR measurements one or more skin-type values indicative of a skin characteristic and/or characterization. In certain embodiments the skin type values are indicative of a Glogau and/or a Fitzpatrick scale value. In certain embodiments the NRM instrument comprises a portable NMR device. In certain embodiments the NMR instrument provides depth resolution of better than 10 μm, preferably better than 5 μm, more preferably better than 3 μm and most preferably better than 2 μm or 1 μm. In certain embodiments the NMR instrument comprises a portable NMR device. In certain embodiments the calculating comprises determining a Fourier transform (e.g., fast Fourier transformation (FFT)) of an NMR signal. In various embodiments the Fourier transformation can be performed by hardware or software. In certain embodiments the calculating comprises determining an NMR signal amplitude as a function of skin depth at a skin location. In certain embodiments the calculating comprises determining an NMR signal amplitude as a function of skin depth where the signal amplitude as a function of skin depth defines a step. In certain embodiments the calculating comprises identifying a step depth (d₀), optionally identifying a step height (Δf), and optionally identifying a step width (σ). In certain embodiments the skin-type values are a function of a step depth (d₀) and/or a step height (Δf), and/or a step width (σ). In certain embodiments the calculating comprises determining a location of a transition between cutis and subcutis and/or a thickness of cutis and/or subcutis. In certain embodiments the NMR measurements comprise measurements of one or more parameters selected from the group consisting of: relaxation time T2 (true), relaxation time T2 (effective), relaxation time T1 (true), relaxation time T1 (effective), self-diffusion coefficient D, signal amplitude, spin modes, pulse sequence CPMG, dipolar encoded longitudinal magnetization, multiquantum build-up, multiquantum decay, diffusion coefficients, chemical shift resolved spectra, component amplitudes in chemical shift resolved spectra, and/or a mathematical function thereof. In various embodiments the methods further involves outputting to a patient medical record the one or more NMR measurements and/or the one or more skin-type values. In various embodiments the methods further involves outputting to a display or printer and/or storing to a computer readable medium the one or more NMR measurements and/or the one or more skin-type values. In certain embodiments the patient medical records comprise one or more of the following: Glogau value for the same skin, Fitzpatrick value for the same skin, skin thickness, skin hardness, skin water content, freckles, scaling, subject identifier, subject age, subject ethnicity, subject gender, and location of skin measurement.

In certain embodiments methods are provided for identifying a skin type for a region of skin of a treatment subject. The methods typically involve providing a skin type database containing skin type records from a plurality of subjects; receiving one or NMR parameters determined from the region of skin and/or skin-type values calculated from said NMR parameters; querying the skin type database using the one or more NMR parameters and/or skin-type values to identify and/or characterize the skin type of the subject; and outputting to a display or printer and/or storing to a computer readable medium a characterization of the skin type for that region of skin of the subject. In certain embodiments the skin-type values are indicative of a skin characteristic or characterization. In certain embodiments the skin-type values are indicative of a Glogau and/or Fitzpatrick scale value. In certain embodiments the NMR parameters are determined using a portable NMR device. In certain embodiments the NMR instrument provides depth resolution of better than 10 μm, preferably better than 5 μm, more preferably better than 3 μm and most preferably better than 2 μm or 1 μm. In certain embodiments the receiving comprises calculating or receiving already a calculated Fourier transform (e.g., a Fast Fourier Transform (FFT)) of an NMR signal depth profile. In certain embodiments the Fourier transformation is performed by hardware or software. In certain embodiments the receiving comprises receiving a measurement of an NMR signal amplitude as a function of skin depth at a location skin location. In certain embodiments the receiving comprises receiving an NMR signal amplitude as a function of skin depth wherein said signal amplitude as a function of skin depth defines a step. In certain embodiments the receiving comprises calculating or receiving already calculated a step depth (d₀), optionally a step height (Δf), and optionally a step width (σ). In certain embodiments the skin-type values are a function of a step depth (d₀) and/or a step height (Δf), and/or a step width (σ). In certain embodiments the skin type values define a location of a transition between cutis and subcutis and/or a thickness of cutis and/or subcutis. In certain embodiments the database is a relational database. In certain embodiments at least a plurality of records in the database comprise NMR data characterizing the skin of a region of a reference subject; and Glogau and/or Fitzpatrick characterization of the same skin of the reference subject. In certain embodiments at least a plurality of records comprise one or more of the following: a step depth (d₀), a step height (Δf), a step width (σ), a location of a transition between cutis and subcutis, a thickness of cutis, a thickness of subcutis, skin thickness, skin hardness, skin water content, skin color, freckles, and scaling. In various embodiments at least a plurality of the skin type records comprise one or more skin NMR parameters selected from the group consisting of proton density, NMR relaxation times, diffusion coefficients, chemical shift resolved spectra, and component amplitudes in chemical shift resolved spectra. In various embodiments at least a plurality of the skin type records comprise one or more skin parameters selected from the group consisting of relaxation time T2 (true), relaxation time T2 (effective), relaxation time T1 (true), relaxation time T1 (effective), self-diffusion coefficient D, or a mathematical function thereof. In various embodiments at least a plurality of the skin type records comprise one or more skin parameters selected from the group consisting of signal amplitude, spin modes, pulse sequence CPMG, dipolar encoded longitudinal magnetization, multiquantum build-up, and multiquantum decay. In various embodiments the skin type records further comprise one or more skin parameters selected from the group consisting of skin thickness, skin hardness, skin water content, freckles, scaling, reference subject identifier, reference subject age, reference subject gender, reference subject ethnicity, and reference subject skin type sample location. In various embodiments receiving one or NMR parameters determined from the region of skin comprises receiving one or more skin NMR parameters selected from the group consisting of proton density, NMR relaxation times, diffusion coefficients, chemical shift resolved spectra, and component amplitudes in chemical shift resolved spectra. In certain embodiments receiving one or NMR parameters determined from the region of skin comprises receiving one or more skin NMR parameters selected from the group consisting of proton density, NMR relaxation times, diffusion coefficients, chemical shift resolved spectra, and component amplitudes in chemical shift resolved spectra. In certain embodiments receiving one or NMR parameters determined from the region of skin comprises receiving one or more skin NMR parameters selected from the group consisting of relaxation time T2 (true), relaxation time T2 (effective), relaxation time T1 (true), relaxation time T1 (effective), self-diffusion coefficient D, or a mathematical function thereof. In certain embodiments receiving one or NMR parameters determined from the region of skin comprises receiving one or more parameters selected from the group consisting of signal amplitude, spin modes, pulse sequence CPMG, dipolar encoded longitudinal magnetization, multiquantum build-up, and multiquantum decay. The method can optionally further comprise receiving one or more skin parameters selected from the group consisting of skin thickness, skin hardness, skin water content, freckles, scaling, a treatment subject identifier, a treatment subject age, a treatment subject gender, a treatment subject ethnicity, and a treatment subject skin type sample location. In certain embodiments the outputting comprises outputting comprises storing to a computer readable medium selected from the group consisting of a magnetic medium, an optical medium, and a flash memory. In certain embodiments the providing a skin type database containing skin type records from a plurality of subjects comprises accessing a remote skin type database.

Also provided is a machine-accessible (e.g., computer readable) medium that provides instructions that, if executed by a machine (e.g., a computer), will cause the machine to perform operations comprising: receiving one or more nuclear magnetic resonance (NMR) parameters and/or skin-type values calculated from the NMR parameters and/or calculating skin-type values from the NMR parameters wherein the NMR parameters are from NMR measurements from skin at one or more locations on a subject and the skin-type values are indicative of a skin characteristic or characterization; and determining and outputting to a display or tangible medium, a treatment plan optimized for a skin type characterization determined from said NMR parameters and/or skin-type values. In certain embodiments the skin-type values are indicative of a Glogau scale value and/or a Fitzpatrick scale value. In certain embodiments the NMR parameters are determined using a portable NMR device. In certain embodiments the NMR instrument provides depth resolution of better than 10 μm, preferably better than 5 μm, more preferably better than 3 μm and most preferably better than 2 μm or 1 μm. In certain embodiments the receiving comprises calculating or receiving already calculated a Fourier transform (e.g., fast Fourier transformation (FFT)) of an NMR signal. In various embodiments the Fourier transformation can be performed by hardware or software. In certain embodiments the receiving comprises receiving a measurement of an NMR signal amplitude as a function of skin depth at a location skin location. In certain embodiments the receiving comprises receiving an NMR signal amplitude as a function of skin depth wherein said signal amplitude as a function of skin depth defines a step. In certain embodiments the receiving comprises calculating or receiving already calculated a step depth (d₀), optionally a step height (Δf), and optionally a step width (σ). In certain embodiments the skin-type values are a function of a step depth (d₀) and/or a step height (Δf), and/or a step width (σ). In certain embodiments the skin type values define a location of a transition between cutis and subcutis and/or comprise a thickness of cutis and/or subcutis. In certain embodiments the method further comprises outputting operation parameters to a treatment device (e.g., a laser, a radiofrequency device, a plasma generator, a pulsed light generator, and the like). In certain embodiments the receiving NMR data comprises receiving previously collected NMR data from an operator, computer readable medium, a patient record, and the like. In certain embodiments the receiving NMR data comprises receiving NMR data from a local or remote NMR device (e.g., while a subject is being scanned, or after the subject has been scanned). In certain embodiments the NMR skin type database comprises a collection of records where at least a plurality of records comprise: NMR data characterizing the skin of a region of a reference subject; and Glogau and/or Fitzpatrick characterization of the same skin of the reference subject. In certain embodiments at least a plurality of the skin type records comprise one or more skin NMR parameters selected from the group consisting of proton density, NMR relaxation times, diffusion coefficients, chemical shift resolved spectra, and component amplitudes in chemical shift resolved spectra. In certain embodiments at least a plurality of the skin type records comprise one or more skin parameters selected from the group consisting of relaxation time T2 (true), relaxation time T2 (effective), relaxation time T1 (true), relaxation time T1 (effective), self-diffusion coefficient D, or a mathematical function thereof. In certain embodiments at least a plurality of the skin type records comprise one or more skin parameters selected from the group consisting of signal amplitude, spin modes, pulse sequence CPMG, dipolar encoded longitudinal magnetization, multiquantum build-up, and multiquantum decay. In various embodiments the skin type records further comprise one or more skin parameters selected from the group consisting of skin thickness, skin hardness, skin water content, freckles, and scaling, subject identifier, reference subject age, reference subject gender, reference subject ethnicity, and reference subject skin type sample location.

In various embodiments, systems are also provided for performing the various methods described herein. One illustrative system comprises a computer processor configured to: receive NMR data obtained from a subject's skin and to calculate a skin-type value from said NMR data and/or to query a database storing a library of NMR skin type characterizations to return one or more skin-type values for said skin, wherein said skin-type values are indicative of a Fitzpatrick and/or Glogau scale value. In certain embodiments the processor or a second processor is configured to generate a treatment plan optimized for a patient skin type characterized by a said NMR data. In certain embodiments the system further comprises an NMR measuring device. In certain embodiments the NMR instrument provides depth resolution of better than 10 μm, preferably better than 5 μm, more preferably better than 3 μm and most preferably better than 2 μm or 1 μm. In certain embodiments the NMR instrument comprises a portable NMR device. In certain embodiments the calculating comprises determining a Fourier transform (e.g., fast Fourier transformation (FFT)) of an NMR signal. In various embodiments the Fourier transformation can be performed by hardware or software. In certain embodiments the system is configured such that the receiving comprises receiving a measurement of an NMR signal amplitude as a function of skin depth at a location skin location and said system is configured to calculate or receive already calculated a step depth (d₀), optionally a step height (Δf), and optionally a step width (σ). In certain embodiments the skin-type value is a function of a step depth (d₀) and/or a step height (Δf), and/or a step width (σ). In certain embodiments the skin type value defines a location of a transition between cutis and subcutis and/or a thickness of cutis and/or subcutis. In certain embodiments the library of NMR skin type characterizations comprises a collection of records wherein at least a plurality of records comprise: NMR data characterizing the skin of a region of a reference subject and/or one or more skin-type values; and, optionally, Glogau and/or Fitzpatrick characterization of the same skin of said reference subject. In certain embodiments at least a plurality of the skin type characterizations comprise one or more skin NMR parameters selected from the group consisting of NMR parameters selected from the group consisting of relaxation time T2 (true), relaxation time T2 (effective), relaxation time T1 (true), relaxation time T1 (effective), self-diffusion coefficient D, signal amplitude, spin modes, pulse sequence CPMG, dipolar encoded longitudinal magnetization, multiquantum build-up, multiquantum decay, diffusion coefficients, chemical shift resolved spectra, component amplitudes in chemical shift resolved spectra, and/or a mathematical function thereof. In certain embodiments at least a plurality of the skin type characterizations further comprise one or more skin parameters selected from the group consisting of skin thickness, skin hardness, skin water content, freckles, scaling, reference subject identifier, reference subject age, reference subject gender, reference subject ethnicity, and reference subject skin type sample location. In certain embodiments the system further comprises means for receiving skin NMR data from a test subject and formulating a query to identify a skin type in said database based on NMR data and/or calculated skin type from said test subject.

In various embodiments methods are also provided for treating a region of interest of the skin of a subject. The methods typically involve identifying a region of interest of the skin of a subject to be treated; making one or more NMR measurements of the region to obtain NMR parameters characterizing the skin region; calculating one or more skin-type values and/or querying an NMR skin type database with the NMR data and/or skin-type values to identify the skin type characterized by the NMR data; calculating and outputting to a display or tangible medium, a treatment plan optimized for the skin type characterization returned from the query; and treating the subject in accordance with the treatment plan. In certain embodiments the calculating comprises analyzing a signal comprising NMR signal amplitude as a function of skin depth at a location skin location to determine a step depth (d₀), optionally a step height (Δf), and optionally a step width (σ). In certain embodiments the calculating comprises calculating a skin-type value that is a function of a step depth (d₀) and/or a step height (Δf), and/or a step width (σ). In certain embodiments the skin type value defines a location of a transition between cutis and subcutis and/or a thickness of cutis and/or subcutis. In various embodiments the treating comprises outputting operation parameters to a treatment device or system (e.g., a laser, a radiofrequency device, a plasma generator, a pulsed light generator, etc.). In certain embodiments the treating comprises selecting a pharmaceutical and/or cosmetic regimen. In certain embodiments the querying comprises utilizing previously collected NMR data entered by an operator or from a computer readable medium, or from a network connection. In certain embodiments the querying comprises utilizing previously collected NMR data from a patient record. In certain embodiments the querying comprises utilizing NMR data from a local or remote NMR device (e.g., while a subject is being scanned, or after the subject is scanned). In certain embodiments the querying further comprises including Glogau and/or Fitzpatrick characterization of the same skin in the query. In certain embodiments the querying further comprises including in the query one or more parameters selected from the group consisting of skin thickness, skin hardness, skin water content, freckles, scaling, subject age, subject gender, subject ethnicity, subject skin type sample location, and measurement depth. In certain embodiments the NMR parameters include one or more parameters as described above. In certain embodiments at least a plurality of the skin type characterizations further comprise one or more skin parameters selected from the group consisting of skin thickness, skin hardness, skin water content, freckles, and scaling.

Methods are also provided for generating a skin type database. The methods typically involve making one or more NMR measurements of a skin region of interest in a reference subject; and storing a plurality of parameters obtained from said NMR measurement(s) and/or one or more skin-type values derived from said NMR measurement(s), in a computer readable medium to form a skin type database. In certain embodiments the skin-type values are indicative of a Glogau and/or a Fitzpatrick scale value. In certain embodiments the method involves determining an NMR signal amplitude as a function of skin depth at a location skin location and calculating a step depth (d₀), optionally a step height (Δf), and optionally a step width (σ). In certain embodiments the skin-type value is a function of a step depth (d₀) and/or a step height (Δf), and/or a step width (σ). In certain embodiments the skin type value defines a location of a transition between cutis and subcutis, and/or a thickness of cutis and/or subcutis. In various embodiments the method further comprises storing Glogau and/or Fitzpatrick characterizations of the same skin region of interesting of the reference subject. In certain embodiments the method further comprises storing the size and/or location of the skin region of interest of the reference subject, and/or the depth at which a measurement is made. In certain embodiments the method further involves storing one or more skin parameters selected from the group consisting of skin thickness, skin hardness, skin water content, freckles, scaling, reference subject identifier, reference subject age, reference subject gender, reference subject ethnicity, reference subject skin type sample location, and sample/measurement depth. In certain embodiments the NMR parameters comprise one or more parameters as described above.

Where NMR measurements are referred to above, it will be appreciated that, in certain embodiments, other quantitative measurements (e.g., positron emission tomography (PET), x-ray, CAT scans, thermography, electrical measurements including for example, conductivity, capacitance, and the like, and various mechanical measurements including stiffness, hydration, and the like) can be substituted therefore, or used in conjunction with NMR measurements.

### DEFINITIONS

When a database is said to contain quantitative parameters (e.g., NMR parameters) it will be understood that the parameters can refer to the actual measured values and/or to values derived from (calculated from) the measured values.

A “skin type database” is a database containing information characterizing skin properties. In various embodiments the skin type database can contain quantitative measurements (e.g., NMR measurements) made of the skin at a particular location on a subject and/or qualitative evaluations (e.g., Glogau and/or Fitzpatrick scale ratings). The database may typically be maintained as a private database behind a firewall within an enterprise. However, this invention is not so limited and the database could actually be made available to the public.

In database terminology, a “record” refers to a collection of information (e.g., as represented by a “row” in a database table). Each record typically contains one or more fields or attributes. A given record may be uniquely specified by one or a combination of fields or attributes known as the record's primary key.

The phrase “providing a skin type database” does not require the actual creation of the database. Providing can simply include accessing such a database (e.g., locally or through a network connection).

The phrase “indicative of a Glogau and/or a Fitzpatrick scale value” indicates that the measured and/or calculated parameter is correlated (preferably at a statistically significant value, e.g., p<0.1, preferably p<0.05, more preferably p<0.01 or 0.005) with a Glogau and/or Fitzpatrick scale value determined for the same skin. (Where the p-value is the probability of obtaining a result at least as extreme as the one that was actually observed, given that the null hypothesis is true).

### BRIEF DESCRIPTION OF THE DRAWINGS

FIG. 1 illustrates the information that can be provided by a one embodiment of a skin type database according to the present invention. For example, a variety of NMR parameters (P₁ . . . P_{K}) can be provided, e.g., as a function of depth in the skin along with corresponding Glogau and/or Fitzpatrick scale values. NMR parameters include, but are not limited to NMR relaxation times, diffusion coefficients, chemical shift resolved spectra, and component amplitudes in chemical shift resolved spectra. In various embodiments the parameters include, but are not limited to relaxation time T2 (true), relaxation time T2 (effective), relaxation time T1 (true), relaxation time T1 (effective), self-diffusion coefficient D, and/or mathematical function(s) thereof.

FIG. 2 illustrates one process by which data for a skin type database according to the present invention may be obtained.

FIG. 3 illustrates the acquisition of quantitative skin type data (e.g., NMR data) at multiple locations (mapping points) on a subject.

FIGS. 4A and 4B illustrate magnets for unilateral NMR sensors with the magnetic field parallel to the sensor surface. FIG. 4A illustrates an early drawing of a u-shaped one-sided NMR sensor (adapted from Matzkanin (1989) Pp. 655-669 In: Nondestructive Characterization of Materials, Springer, Berlin). FIG. 4B illustrates the magnet arrangement for the Profile NMR-MOUSE which provides a constant gradient in the y-direction and a constant field |B₀| in the xz-plane at one particular depth y (see, e.g., Perlo et al. (2005) J. Magn. Reson., 176: 64-70).

FIG. 5 illustrates a network system 230 suitable for storing and retrieving information in skin type databases of the present invention.

FIG. 6 schematically illustrates various software documents and entities employed by the client server network of FIG. 5 to provide skin type information in response to user queries.

FIGS. 7A and 7B illustrate certain embodiments of record types comprising a skin type database.

FIG. 8 shows a block diagram illustrating one embodiment of a system incorporating a skin type database according to the present invention.

FIG. 9 shows one process that provides a method of treatment utilizing a skin type database according to the present invention.

FIG. 10 illustrates facial positions identified for measurement of NMR depth profiles. Profiles were acquired for positions one to seven and averaged upon validation of data quality.

FIG. 11 illustrates the measurement set-up used in Example 1. The Profile NMR-MOUSE® consists of a U-shaped magnet with a radio-frequency (rf) coil in the gap. The sensitive slice is located above the sensor surface. The NMR-MOUSE® is mounted on a step-motor driven lift that moves it up and down changing the distance between the patient skin and the sensor surface. The lift is positioned underneath an examination table. A copper cloth was used as an rf shield to reduce external noise.

FIG. 12 illustrates facial skin depth profiles for one volunteer. The profiles are assigned to the seven measurement positions identified in FIG. 10.

FIGS. 13A and 13B illustrate determination of fit parameters (FIG. 13A) and division of the parameter distribution into bins by example of the distribution of step height Δf (FIG. 13B).

FIG. 14 illustrates the joint probability densities W(x_{G}, Pᵢ) and W(x_{F}, Pᵢ) of fit parameters Pᵢ and skin type ratings according to Glogau and Fitzpatrick. Trends are indicated by arrows.

FIG. 15 shows a representation of the longitudinal relaxation time T₁ and the short effective transverse relaxation time T_{2eff} of the subcutaneous fillers listed in Table 3 the respective measurement uncertainties.

FIG. 16 illustrates depth profiles into the skin of the arm of a volunteer with and without filler. The signals of the pure filler are also given.

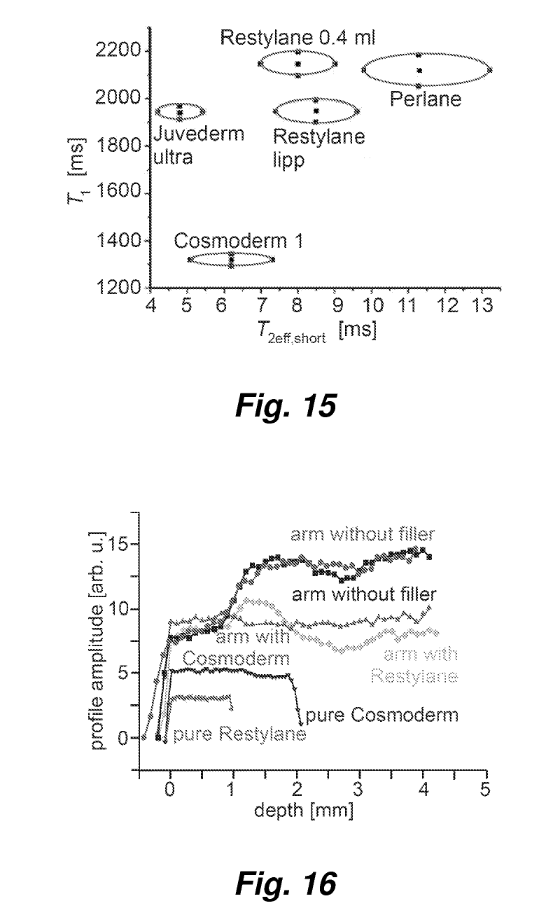

### DETAILED DESCRIPTION

In various embodiments this invention pertains to the discovery that objective/quantitative measures of various parameters of skin (e.g., NMR parameters, MRI data, PET data, conductivity, resistance, capacitance, etc.) can be used to characterize the “skin-type” in a manner that is useful in the creation/design of skin treatments, in evaluating cosmetics, in planning surgical alterations, and the like. In certain embodiments, nuclear magnetic resonance (NMR) skin measurements are made that can be correlated with the Fitzpatrick (Fitzpatrick et al. (1963) Dermatol Wochenschr 147: 481-489) and/or Glogau scales (Glogau (1996) Semin Cutan Med Surg, 15(3): 134-138) typically used to evaluate/characterize skin types. Unlike the Fitzpatrick and/or Glogau scales that can be highly subjective in their application, the use of quantitative parameters and/or “skin-type” values derived therefrom can reduce or eliminate the subjective component of the evaluation rendering methods of skin-type characterization more widely available, more uniform in application, and more easily taught to practitioners.

Accordingly, in certain embodiments, methods are provided for characterizing skin type in a subject (e.g., in a human), where the methods involve performing nuclear magnetic resonance (NMR) measurements of the region(s) of skin of interest. The NMR measurements can be used directly in a skin-type characterization and/or they can be used to calculate one or more skin-type values that characterize the measured skin-type. In certain embodiments the skin type values so calculated are indicative of (e.g., correlated to) a corresponding Glogau and/or Fitzpatrick scale value and/or range and so are readily adapted to use by physicians, researchers, etc.

In addition to providing objective measurements and characterization of skin-types, in various embodiments this invention pertains to the creation and/or use of a “skin type” database that relates objective/quantitative indicia characterizing skin properties to more traditional “subjective scales” (e.g., the Glogau scale, the Fitzpatrick scale, etc.) used to characterize skin types. Such a database finds a number of uses, for example in cosmetic and clinical dermatology, plastic surgery, and aesthetic surgery.

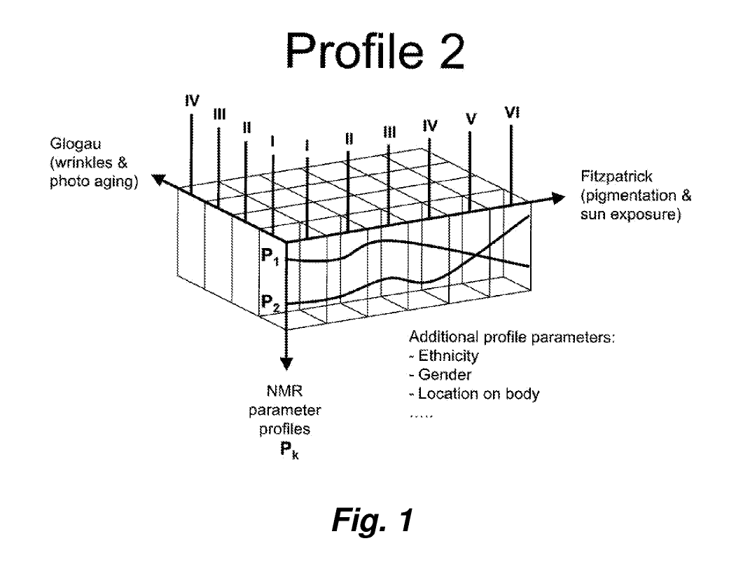

In certain embodiments, the skin type database typically comprises records containing objective/quantitative data (e.g., NMR parameters, MRI data, PET data, etc.) and/or skin-type values derived from such data, measured for different skin types often measured at different locations on the body for subjects of different age, gender, ethnicity, health condition, and the like (see, e.g., FIG. 1). In various embodiments, the skin type database can also contain qualitative evaluations of the same skin “samples” providing, for example measures of the skin according to the Glogau scale (see, e.g., Glogau (1996) Semin Cutan Med. Surg. 15(3): 134-138), the Fitzpatrick scale (see, e.g., Fitzpatrick et al. (1963) Dermatol Wochenschr 147: 481-489), and the like. In various embodiments, the database is linked to and/or includes annotation data containing additional information (e.g., patient identifier, health status, treatment history, etc.) regarding the subject from which the skin type data is obtained. In various embodiments, the database is linked to and/or includes data from patient medical records and/or insurance records.

In certain embodiments, the database can be provided as a “stand alone” database, mounted, for example on a single computer or on a single computer comprising a treatment planning or treatment management system. In certain other embodiments, the database is provided on a network server (e.g., a server for an intranet or internet) so that it can be queried by one or more remote “clients”.

In use, the skin type database can be used by dermatologists, plastic surgeons, aesthetic surgeons, cosmetologists, skin care businesses, and the like. For example, dermatologists can measure skin parameters in a patient, using, for example, an NMR probe, calculate relevant skin-type values and/or reference measured parameters by querying the skin type data base, and select the skin treatment procedure accordingly. An example is the heat treatment of aged skin to enhance the collagen production by radiofrequency, plasma, fractional carbon dioxide laser, long wave monochromatic laser, or broad spectrum intense pulsed light, singly or in combination. The particular heat treatment and parameters by which it is administered can be determined in part by the skin-type characterization.

Apart from clinics, hospitals, and doctors offices, the measurement methods, and/or databases and/or methods of use described herein will find use by skin care companies, companies that produce skin care devices including lasers, radiofrequency devices, plasma generators, intense pulsed light generators, and manufacturers of magnetic resonance instruments like (e.g., General Electric, Philips Medical Systems, Siemens Medical Solutions, and smaller instrument manufacturers).

In one illustrative embodiment, high-resolution skin depth profiles as well as skin parameters at selected depths from subjects are collected by NMR and similar devices. The NMR measurements can be used directly to calculate skin-type values that, in certain embodiments, are indicative of Glogau and/or Fitzpatrick scale values or ranges. In various embodiments the NMR measurements can be used, optionally in conjunction with the skin-type values, to establish a database of skin maps of the human body, in particular of the human face. Parameters of interest include, for example, the proton density, NMR relaxation times, diffusion coefficients, chemical-shift resolved spectra, and component amplitudes in such spectra. Also of interest are derived skin-type values including for example, but not limited to, a depth profile of an NMR signal, the existence location of a step in such a profile and optionally, a characterization of such a step (e.g., a step depth (d₀), and/or a step height (Δf), and/or a step width (σ)) when present. In addition to such “quantitative” data, qualitative data including, but not limited to Glogau scale and/or Fitzpatrick scale evaluations, degree of scaling, freckling, and the like, can also be determined.

In certain embodiments when a patient undergoes treatment, skin parameters, e.g., for the treatment area, are collected by NMR, and/or MIR, and/or PET, and/or capacitance meter, and/or ohmmeter, and/or other quantitative devices. Using the parameters determined by the measurement(s), skin-type values are calculated and/or the skin type database is queried for to provide a skin characterization, and, in certain embodiments, a recommended treatment protocol. The skin characterization informs and guides the selection of appropriate treatment strategies. The measurements combined with the skin type database thus provide a “calibration map” for the patient.

Simple calibration maps can readily be used to gage many treatment strategies. In addition, quantitative parameter maps can enhance and expand the use of such maps as a medical reference source, to predict adverse effects, and the like. Different levels of detail will serve the needs of different skin treatments.

In certain embodiments the measurements of subjects and the calculation of skin-type values and/or use of a skin type database, can be used to predict age-related changes in appearance/skin type. Multiple measurements of different areas of, for example, the face can produce 1) A generic face map; 2) Face maps of people grouped according to gender, age, race, lifestyle, and the like; and 3) Individual face maps of personal identity. The process can be expanded to also provide calibrated “body maps”.

Thus, in various embodiments, this invention provides methods of calculating skin-type values from quantitative skin measurements (e.g., NMR, PET, MRI, etc.), computer readable media containing instructions to perform such calculations, microprocessors and/or systems programmed to perform such calculations, methods of creating and populating a skin type database, computer readable media comprising such a database, systems, systems coupled to or incorporating such a skin type database, as well as methods of use thereof. In certain embodiments the methods of use include, for example, a method of identifying a skin type for a region of skin of a treatment subject, where the method involves providing a skin type database containing skin type records from a plurality of subjects; receiving one or more NMR (and/or other) parameters determined from the region of skin and/or receiving or calculating skin-type values from the NMR (and/or other) parameters; querying the skin type database using the one or more NMR (and/or other) parameters, and/or skin type values, to identify the skin type of the subject; and, optionally, outputting to a display and/or printer and/or treatment device, and/or storing to a computer readable medium a characterization of the skin type for the region of skin of the subject. In another embodiment methods are provided for treating a region of interest of the skin of a subject, where the method involves identifying one or more regions of interest of the skin of a subject to be treated; making one or more NMR (or MRI, PET, etc.) measurements of said region to obtain NMR (or other) parameters characterizing the skin region; optionally receiving already calculated or optionally calculating from the NMR data skin type values, directly using the NMR data and/or calculated skin-type values and/or querying an NMR skin type database with the NMR parameters and/or calculated skin-type values to identify the skin type characterized by said NMR (or other) data; calculating and outputting to a display and/or computer readable medium and/or treatment device, a treatment plan optionally characterized by and/or optimized for the skin type characterization returned from said query and/or calculated directly from the data; and treating the subject in accordance with the treatment plan.

While quantitative skin measurements are frequently described herein with respect to nuclear magnetic resonance (NMR) measurements other “quantitative” or semi-quantitative measurement methods (e.g., PET, MRI, skin conductivity, skin capacitance, temperature maps, etc.) are also contemplated.

I. Measuring Skin-Type and/or Providing/Populating a Skin-Type Database.

In various embodiments the skin of one or more subjects is measured at one or more locations using one or more quantitative measurement methods (e.g., NMR, PET, MRI, etc.) to provide measured parameters for the skin at the measured location(s). The parameters can be used directly to characterize the skin at the measured locations and/or skin type values characterizing the skin can be calculated from the measured parameter(s), and/or the measured parameters and/or skin type values can be used in conjunction with a skin-type database to characterize the skin. In certain embodiments the values determined thereby are entered into a patient record. In certain embodiments the skin characterization can be used in treatment planning, by dermatologists, plastic surgeons, aesthetic surgeons and the like. The skin characterization can also be used by researchers, cosmetologists, and the like.

In various embodiments a skin type database is created by scanning/measuring a plurality of subjects at different regions of the skin to determine one or more “quantitative” skin type parameters. The measured skin type parameters (or data derived therefrom such as skin-type values) can be input and stored as records in a skin type database. In various embodiments skin type parameters are characterized by one or more of the following: the location on the body where the measurement is made, the area over which the measurement was made, the age of the subject, the gender of the subject, the ethnicity of the subject, and the like. In certain embodiments “qualitative” parameters are evaluated and input into the database. Such qualitative parameters include, but are not limited to a Glogau scale rating and/or a Fitzpatrick scale rating. In certain embodiments the records contain or are linked to data records containing one or more of the following: a patient/subject identifier, an evaluation of previous treatment, counter indications, skin sensitivities, and the like.

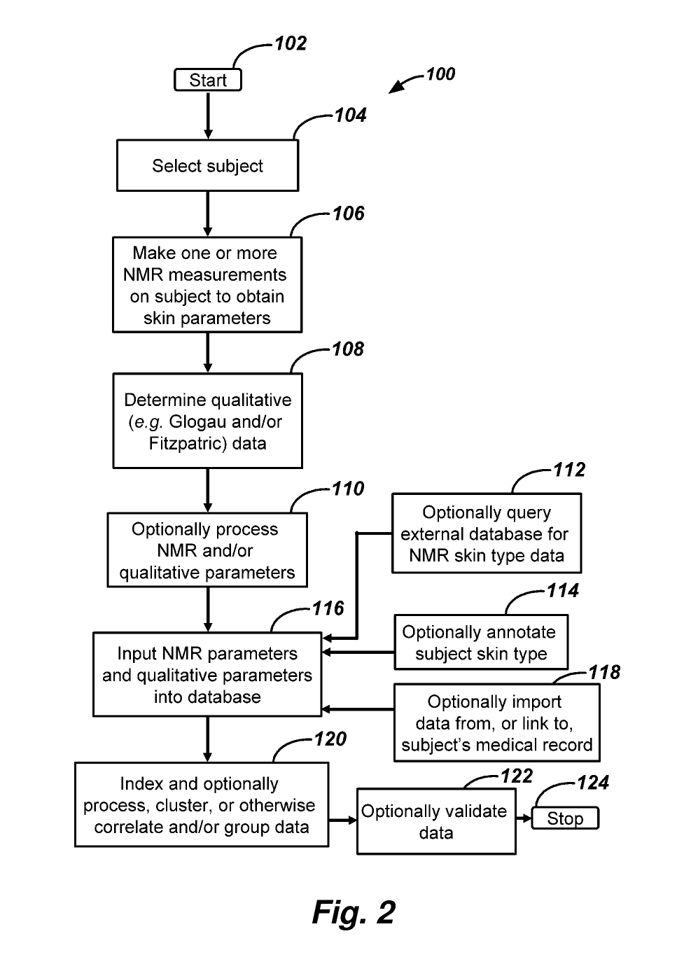

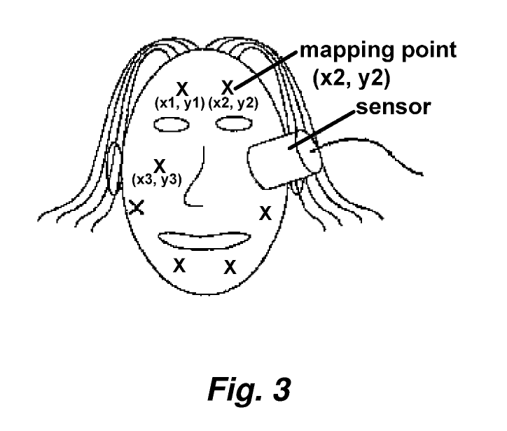

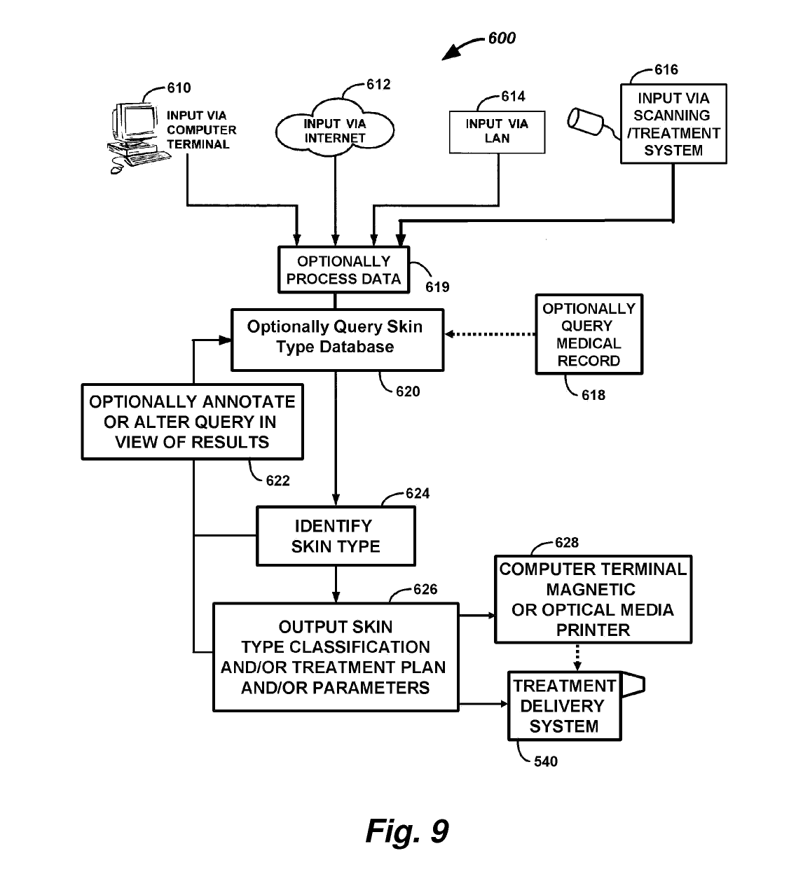

The following description presents one process by which data for a skin type database according to the present invention may be obtained. The process is illustrated in FIG. 2. In the process flow of FIG. 2, the process begins at 102. As represented by step 104 subject is identified/selected 104, and, as shown in step 106, one or more NMR (or other quantitative) measurements are made, typically at a plurality of locations (mapping points), e.g., as illustrated in FIGS. 3 and 10. In addition, as illustrated in optional step 108, various qualitative evaluations of the skin (e.g., Glogau index, Fitzpatrick index, etc.) can also be made. The quantitative and/or qualitative values determined for the subject can be left in their “raw” state or can optionally be processed as shown in step 110. The processing can involve any of a number of operations, including for example, performing Fourier transformations of the raw data, averaging multiple measurements, correlating measurements, fitting curves, normalizing data, calculating measures of variability, clustering or discriminating measurements, and the like.

In certain embodiments, the processing involves identifying a step in a profile of signal amplitude as a function of skin depth. In certain embodiments the step is characterized by a depth and/or a step height, and/or a step width.

The raw and/or processed measurements/parameters can then be input into one or more records of a database as illustrated in step 116. In addition to, or alternatively to, selecting and measuring a subject to populate the database, data can also be obtained from external databases containing similar information. Thus, for example, where external databases are available containing, for example NMR skin type data and associated Glogau and/or Fitzpatrick scale information, the external database can be queried and the data from that database also input into the subject skin type database, as illustrated in steps 112 and 116. Thus, in certain embodiments, the database(s) of the present invention may contain skin type data obtained for example from a number of sources, including data from external sources, such as public databases where available, submissions from independent researchers, and the like. In addition, enterprise skin type data, that is, proprietary data obtained and processed by the database developer is generally used.

Following or during acquisition of the relevant/desired measurements, the data can be loaded into a database, as represented by step 116 in process 100. As illustrated by step 120 in process flow 100, after or while the data is input (loaded) into the database, records can be indexed, and data can optionally be processed, optionally clustered and/or otherwise analyzed, correlated and/or grouped. The entered data can also, optionally, be validated 122. In certain embodiments, the database is a relational database and includes, for example, a “skin-type module” and, optionally, an “annotation module” and/or a “record linking module”. The skin type module inputs and/or stores unannotated skin type data, provided, for example, as NMR parameters determined from a particular subject at particular locations and/or skin-type values derived therefrom. The annotation module can identify the NMR records by reference IDs, and can include annotated information regarding each of the NMR measurements and/or skin-type values. In certain embodiments the annotations can include, for example, information about the age, gender, ethnicity, health status of the subject, treatment history, and the like. A “record-linking module” can import or link to data in the subject's health record. The process concludes at 124.

A number of computer platforms can be used to perform the necessary calculations for various algorithmic processes employed in the data processing procedure illustrated in flow 100 (e.g., obtaining quantitative and qualitative measurements, annotating records, linking to medical records, etc.). For example, a number of computer workstations from a variety of manufacturers can be used. For example, workstations produced by Silicon Graphics, Inc. (SGI) of Mountain View, Calif., and Apple Computer (e.g., MACPRO®), are suitable for performing such operations.

A) Obtaining NMR Data.

As explained above, in various embodiments the methods described herein involve obtaining quantitative data characterizing skin at one or more locations on a subject. The quantitative data can include data determined from various detection methods including, but not limited to nuclear magnetic resonance (NMR), positron emission tomography (PET), x-ray, CAT scans, thermograph, resistance, capacitance, and the like.

In one illustrative embodiment, as shown in Example 1, the skin is measured using nuclear magnetic resonance. This can be used to characterize the skin of the measured subject, and/or in certain embodiments to populate a skin type database. In various embodiments the measurements can be made of the entire skin thickness, or can be at various depths in the skin, and/or can provide a profile of various parameters as a function of skin depth.

In various embodiments, the NMR data is acquired using a single-sided NMR probe. The ability to perform magnetic resonance measurements by simply placing a sample on the surface of the RF probe or the RF probe against a sample (e.g., skin) surface (so-called single-sided NMR) is a rather attractive method for obtaining the skin NMR measurements for the skin type database. A fundamental contribution to single-sided NMR development was made by Prof. Bernard Blümich's group, where in 1996 a prototype for the mobile surface scanner, appropriately named the NMR-MOUSE® was developed (see, e.g., Eidmann et al. (1996) J. Magnetic Resonance, Series A, 122(1): 104-109; and www.nmr-mouse.de).

The basic NMR-MOUSE® is a palm-size NMR device that can be built up from two permanent magnets. In various embodiments they are mounted on an iron yoke with anti-parallel polarization to form the classical horseshoe geometry. The main direction of the polarization field B₀ is across the gap. The rf field B₁ is generated by a surface coil which is mounted in the gap. Therefore, B1 is also inhomogeneous. Despite the field inhomogeneties, relaxation rates are accessible. In various embodiments measurements can be performed using 1H-NMR.

Commercial devices are available that incorporate the NMR-MOUSE and other single-sided probe variants. Thus, for example, MINISPEC PROFILER® (see, e.g., Bruker Optics Inc—Minispec Division, The Woodlands, Tex., USA) is a low-cost NMR instrument that reduces the spatial restrictions of the sample size in conventional NMR experiments. The mq-ProFiler is a compact NMR relaxometer, equipped with single-sided magnet and RF probes for performing 1H-NMR experiments within the first few millimeters below the surface of arbitrarily shaped samples. The system is based on Bruker's minispec mq-BB console, a broadbanded electronics unit which works with any kind of minispec probe. In one embodiment, the magnet assembly of the mq-ProFiler consists of two rectangular magnets, placed in an antiparallel configuration and fixed to an iron yoke, and generates a static surface field. The measurement depth can be selected by simply exchanging the RF inserts. An insert-specific preset parameter table (optimal pulse length, operating frequency, etc.) is loaded in the minispec software, and no other set-up actions are required.

In addition, apparatus, systems, and methods for compact single-sided NMR measurements are described in U.S. Pat. Nos. 6,489,767, and 6,977,503, which are incorporated herein by reference for all purposes.

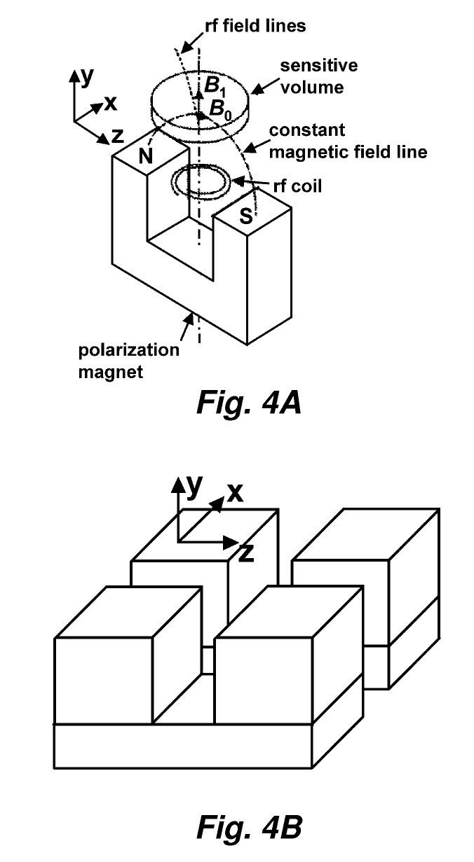

While resolution of different layers of skin is somewhat reduced or limited with the curved sensitive volume of the original, U-shaped NMR-MOUSE™ (Guthausen et al. (1998) Pp. 195-209 In: NMR in Chemical Engineering, Stapf & Han, eds., Wiley-VCH, Weinheim) and the Minispec PROFILER®, the Profile NMR-MOUSE® (see, e.g., FIGS. 4A and 4B) (Perlo et al. (2002) J. Magn. Resonan. 176: 64-70; U.S. Patent Publication: 2002/0079891) provides the flat and thin sensitive volume that enhances such measurements in addition to convenient access to nearly all parts of the human body (see also: U.S. Patent Publication 2007/0182413, which is incorporated herein by reference).

In certain embodiments measurement time is reduced by the use of a high-resolution NMR depth profiler that can measure the Fourier transform of the depth profile (see, e.g., Perlo et al. (2005) J. Magn. Reson., 176: 64-70; Meiboom and Gill (1958) Rev. Sci. Instrum., 29: 688-691, and the like).

In various measurements of depth profiles through human skin, contrast can be adjusted by proper choice of the parameter w (the ratio of two definite integrals of the echo envelope (see, e.g., Blümich et al. (2005) Acta Physica Polonica A, 108: 13-23), or the signal amplitudes and relaxation times are determined from exponential fits of the CPMG decays. Profiles through the palm of the hand demonstrate the excellent reproducibility of the measurements. From the shape of the profile, different skin layers can be discriminated and assigned to the stratigraphy of the skin.

In various embodiments, one or more NMR parameters characterizing one or more features of the skin are determined. The parameters can be determined for the entire skin thickness at a particular location or can be determined as a function of depth into the skin.

Suitable NMR parameters of interest include, but are not limited to one or more of the following: proton density, NMR relaxation times, diffusion coefficients, chemical shift resolved spectra, and component amplitudes in chemical shift resolved spectra. In certain embodiments NMR parameters of interest include, but are not limited to one or more of the following: relaxation time T2 (true), relaxation time T2 (effective), relaxation time T1 (true), relaxation time T1 (effective), self-diffusion coefficient D, or a mathematical function thereof. In certain embodiments NMR parameters of interest include, but are not limited to one or more of the following: signal amplitude, spin modes, pulse sequence CPMG, dipolar-encoded longitudinal magnetization, multiquantum build-up, multiquantum decay, and the like.

While the foregoing discussion has been directed to single-sided NMR, the use of other NMR methods including, but not limited to conventional low-field NMR, NMR tomography, and the like are not excluded. Thus, for example, other approaches to in vivo NMR measurement of skin can utilize tomographs fitted with surface gradient coils and surface coils to obtain an acceptable depth resolution of 70 p.m (see, e.g., Richard et al., (1993) J. Invest. Dermatol., 100: 705-709; Richard et al. (1991) J. Invest. Dermatol., 97: 120-125). Also, the stray-field technology with the GARfield magnet has been used to study the skin in vitro and in vivo of body extremities like the finger or the arm which are compatible with geometrical constrains imposed by the semi-open magnet geometry (see, e.g., Mitchell et al. (2006) Prog. Nucl. Magn. Reson. Spectr., 48: 161-181; Bennett et al. (2003) Magn. Reson. Imag., 21: 235-241); Doughty et al. (2006) Pp. 89-107 In: NMR in Chemical Engineering, Stapf & Han, eds., Wiley-VCH, Weinheim; Backhouse et al. (2004) J. Pharm. Sci., 93: 2274-2283; McDonald et al. (2005) J. Pharm. Sci., 94: 1850-1860); Dias, et al. (2003) J. Phys. D: Appl. Phys., 36: 364-368) with a high depth resolution of up to 5 JAM comparable to that of the Profile NMR-MOUSE™ (Casanova et al. (2006) Pp. 107-123 In: NMR in Chemical Engineering, Stapf & Han, eds., Wiley-VCH, Weinheim).

While the foregoing description focused on NMR measurements, it will be recognized that other measurement methods can be used in combination with or as a substitute for various NMR parameters. Such measurements include, but are not limited to positron emission tomography (PET), x-ray, CAT scans, thermography, electrical measurements including for example, conductivity, capacitance, and the like, and various mechanical measurements including stiffness, hydration, and the like.

B) Calculating Skin-Type Values.

In various embodiments the measured quantitative skin parameters are used directly to characterize skin type, to populate and/or query a skin-type database, and/or in treatment planning and the like. In various embodiments, values derived/calculated from the measured parameters can be used in addition to or instead of the directly measured values.

In certain embodiments the derive/calculated skin type values are indicative of the Glogau and/or Fitzpatrick scale values for the measured skin. In one illustrative embodiment, as shown in Example 1, skin depth profiles are analyzed by fits with a convolution of a heaviside step function and a Gauss function. Three illustrative parameters extracted from the fit: the position d₀ of the step, the standard deviation a defining the width of the step, and the step height Δf can be correlated with the Glogau and Fitzpatrick ratings of the subject's skin.

This calculation is intended to be illustrative and not limiting. Using the methods described herein numerous other derived values can be calculated by one of skill and the invention need not be limited to the use of a particular derived or calculated value. Moreover it is recognized that, in certain embodiments, the derived values need not be significantly correlated with Glogau and/or Fitzpatrick scale values for the measured skin and may themselves provide a better (e.g., more accurate, more reproducible) skin type characterization than these scale values. For example, by way of illustration such derived values may be correlated with other quantitative parameters such as skin hydration, skin thickness, a location of a transition between cutis and subcutis, thickness of cutis and/or subcutis, hydration of cutis and/or subcutis, skin conductivity, skin capacitance, skin pigmentation, and the like.

C) Obtaining Glogau and/or Fitzpatrick Data.

In certain embodiments, the skin type database is also populated with “qualitative” measurements/characterizations of the skin to accompany the quantitative measurements. Two widely used qualitative scales are the Fitzpatrick scale (see, e.g., Fitzpatrick et al. (1963) Dermatol Wochenschr 147: 481-489) and the Glogau Photoaging scale (see, e.g., Glogau (1996) Semin Cutan Med Surg, 15(3): 134-138).

The Fitzpatrick scale has been widely used to characterize skin types, but it only takes into account the skin's pigmentation and reaction to sun exposure (see, e.g., Table 1).

TABLE 1
Fitzpatrick classification of skin. The classification denotes 6
different skin types, skin color, and reaction to sun exposure.

| Group | Description |
|---|---|
| I | Very white or freckled -- Always burn. |
| II | White -- Usually burn. |
| III | White to olive -- Sometimes burn. |
| IV | Brown -- Rarely burn |
| V | Dark Brown -- Very rarely burn. |
| VI | Black -- Never burn. |

The “Glogau Photoaging scale” divides skin into four types according to the amount of wrinkles that are present (see, e.g., Table 2, and Glogau (1996) Semin Cutan Med Surg, 15(3): 134-138). There are no widely accepted skin typing systems that take into account wrinkles, pigmentation, dryness and

TABLE 2
Glogau classification of aging.

| | | Typical | | |
|---|---|---|---|---|
| Group | Classification | Age | Description | Skin Characteristics |
| I | Mild | 28-35 | No wrinkles | Early photoaging |
| | | | | mild pigment changes |
| | | | | no keratosis |
| | | | | minimal wrinkles |
| | | | | minimal or no makeup |
| II | Moderate | 35-50 | Wrinkles in | Early to moderate photoaging |
| | | | motion | early brown spots visible |
| | | | | keratosis palpable, but not visible |
| | | | | parallel smile lines begin to appear |
| | | | | wears some foundation |
| III | Advanced | 50-65 | Wrinkles at | Advanced photoaging |
| | | | rest | obvious discolorations |
| | | | | visible capillaries (telangiectasias) |
| | | | | visible keratosis |
| | | | | wears heavier foundation always |
| IV | Severe | 60-75 | Only wrinkles | Severe photoaging |
| | | | | yellow-gray skin color |
| | | | | prior skin malignancies |
| | | | | wrinkles throughout-no normal skin |
| | | | | cannot wear makeup because it cakes |
| | | | | and cracks |

One of ordinary skill in the art will readily understand how to determine Fitzpatrick and/or Glogau scale metrics.

D) Annotating the Database.

In various embodiments, the skin type database records are additionally annotated to include, for example, additional descriptive information regarding the subject, and/or the measured skin samples. For example, such annotations can include a subject identifier, age, gender, marital status, history of exposure to sun and/or radiation sources (e.g., UV radiation), previous or current therapies, current or previous cosmetic and/or therapeutic regimen, disease history, history regarding reconstructive surgery, ablative therapies, and the like, history regarding cancer occurrence and/or therapy, information regarding chemical and/or drug allergies or sensitivities, and the like. This list is meant to be illustrative and not limiting.

The database record(s) can be annotated by the action of a user manually entering the data. In certain embodiments, the data can be entered while taking a patient history. In certain embodiments, the data can be provided by linking the skin type database to a patient record database and/or importing data from a patient record database.

### II. The Database Environment.

In certain embodiments, the database can be provided as a “stand alone” database, mounted, for example on a single computer or on a single computer comprising a treatment planning or treatment management system. In certain other embodiments, the database is provided on a network server (e.g., a server for an intranet or internet) so that it can be queried by one or more remote “clients”.

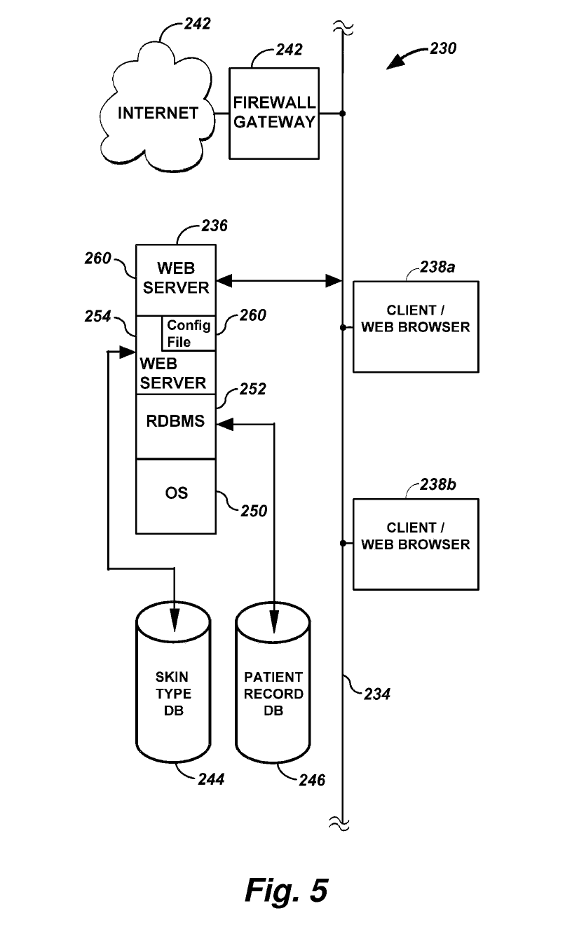

FIG. 5 depicts a network system 230 suitable for storing and retrieving information in relational databases of the present invention. Illustrated network 230 includes a network link 234 (e.g., network cable, wireless network, etc.) to which a network server 236 and clients 238a and 238b (representative of possibly many more clients) are connected. Network link 234 can also be connected to a firewall/gateway 240 which is in turn connected to the Internet 242.

Network 230 can be any one of a number of conventional network systems, including, for example, a local area network (LAN) or a wide area network (WAN), as is known in the art (e.g., using Ethernet, IBM Token Ring, or the like). The network can include functionality for packaging client calls in a well-known format (e.g., URL) together with any parameter information into a format (of one or more packets) suitable for transmission across a network link 234 (e.g., cable or wireless), for delivery to database server 236.

In various embodiments server 236 includes the hardware necessary for running software to (1) access skin type database data for processing user requests, and (2) provide an interface for serving information from or to client machines 238a, 238b, etc. In certain embodiments, the software running on the server machine supports the World Wide Web protocol for providing page data between a server and client.

Client/server environments, database servers, relational databases and networks are well documented in the technical, trade, and patent literature. For a discussion of database servers, relational databases and client/server environments generally, and SQL servers particularly, see, e.g., Nath, A., The Guide To SQL Server, 2nd ed., Addison-Wesley Publishing Co., 1995 (which is incorporated herein by reference for all purposes).

As shown, server 236 includes an operating system 250 (e.g., UNIX, LINUX, WINDOWS, OS10, etc.) on which runs a relational database management system 252, a World Wide Web (e.g., Web II) application 254, and a World Wide Web server 256. The software on server 236 may assume numerous configurations. For example, it may be provided on a single machine or distributed over multiple machines.

In various embodiments, world wide web application 254 includes the executable code necessary for generation of database language statements (e.g., Standard Query Language (SQL) statements). Generally, the executables will include embedded SQL statements. In addition, application 254 can include a configuration file 260 that contains pointers and addresses to the various software entities that comprise the server as well as the various external and internal databases which are accessed to service user requests. Configuration file 160, when present, can also direct requests for server resources to the appropriate hardware—as may be necessary should the server be distributed over two or more separate computers.

In various embodiments each of clients 238a and 238b can include a World Wide Web browser, or other executable, for providing a user interface to server 236. Through the Web browser, or other executable, clients 238a and 238b construct search requests for retrieving data from a skin type database 244 often in conjunction with data (e.g., NMR data) provided by scanning/measuring a subject, and optionally in conjunction with information from patient record database 246. Thus, the user will typically point and click to user interface elements such as buttons, pull down menus, scroll bars, etc., conventionally employed in graphical user interfaces. The requests so formulated with the client's Web browser (or other executable) are transmitted to Web application 254 which formats them to produce a query that can be employed to extract the pertinent information from the skin type database 244 optionally in conjunction with data from the patient record database 246.

In certain embodiments, for example, in a patient treatment system, clients 238a and 238b can be included as components of a treatment device where acquisition of data from a patient automatically generates the request/query to the server essentially without user intervention.

In the embodiment, illustrated in FIG. 5, the Web application accesses data in skin type database 246 by first constructing a query in a database language (e.g., Sybase or Oracle SQL). The database language query is then handed to relational database management system 252 which processes the query to extract the relevant information from database 246. In the case of a request to access skin type database 244, Web application 254 cam communicate the request to that database without employing the services of database management system 252.

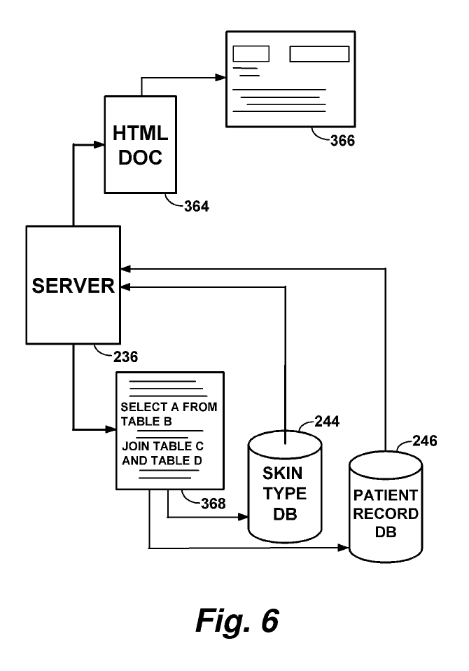

The procedure by which user requests are serviced is further illustrated with reference to FIG. 6. In this embodiment, the World Wide Web server component of server 236 provides Hypertext Mark-up Language documents (“HTML pages”) 365 to a client machine. At the client machine, the HTML document provides a user interface 366 which is employed by a user to formulate his or her requests for access to database 246. That request is converted by the Web application component of server 236 to a SQL query 368. That query is used by the database management system component of server 236 to access the relevant data in database 244, and optionally patient record database 246 and provide that data to server 236 in an appropriate format. Server 236 then generates a new HTML document relaying the database information to the client as a view in user interface 366.

While the embodiment shown in FIG. 6 employs a World Wide Web server and World Wide Web browser for a communication between server 236 and clients 238a and 238b, other communications protocols will also be suitable. For example, client calls may be packaged directly as SQL statements, without reliance on Web application 254 for a conversion to SQL.

When network 230 employs a World Wide Web server and clients, it typically supports a TCP/IP protocol. Local networks such as this are sometimes referred to as “Intranets.” An advantage of such Intranets is that they allow easy communication with public domain databases residing on the World Wide Web. Thus, in certain embodiments of the present invention, clients 238a and 138b can directly access data (via Hypertext links for example) residing on Internet databases using a HTML interface provided by Web browsers and Web server 236.

If the contents of the local databases are to remain private, a firewall 242 preserves in confidence the contents of the skin type database 244 and/or patient record database 246.

In certain embodiments skin type database 244 is a flat file database including separate partitions for skin type data from different subjects.

In most typical embodiments, however, the information in the skin type database 244 is stored in a relational format. Such a relational database supports a set of operations defined by relational algebra. It generally includes tables composed of columns and rows for the data contained in the database. Each table has a primary key, being any column or set of columns the values of which uniquely identify the rows in the table. The tables of a relational database may also include a foreign key, which is a column or set of columns the values of which match the primary key values of another table. A relational database is also generally subject to a set of operations (select, project, product, join and divide) which form the basis of the relational algebra governing relations within the database. As noted above, relational databases are well known and documented (see, e.g., Nath, A., The Guide To SQL Serve, referenced above).

A relational database may be implemented in different ways. In ORACLE® databases, for example, the various tables are not physically separated, as there is one instance of work space with different ownership specified for different tables. In SYBASE® databases, in contrast, the tables may be physically segregated into different “databases.”

One specific configuration for network 230 for multiple users provides both the skin type database and annotation database and/or patient record database on the same machine. If there is a high volume of sequence searching, it may be desirable to have a second processor of similar size and split the application across the two machines to improve response time.

A suitable dual processor server machine may be any of the following workstations: SUN—ULTRA-SPARC 2® (Sun Microsystems, Inc. of Mountain View, Calif.), SGI—CHALLENGE® (Silicon Graphics, Inc. of Mountain View, Calif.), and DEC—2100A® (Digitial Electronics Corporation of Maynard, Mass.). Multiprocessor systems (minimum of 4 processors to start) may include, but are not limited to, the following: Sun—ULTRA SPARC ENTERPRISE 4000® SGI—CHALLENGE XL®, DEC—8400®, and Apple MAC PRO®.

In various embodiments the network can include a 10-base-T connection, be TCP/IP capable, and provide access to Internet.

While the skin-type database is described above—with respect to a networked client—server architecture, it will be recognized, that in certain embodiments, the database can be mounted in a single device. In various embodiments the device can be a stand-alone computer, a treatment planning system, and the like.

### III. Model of the Skin Type Database.

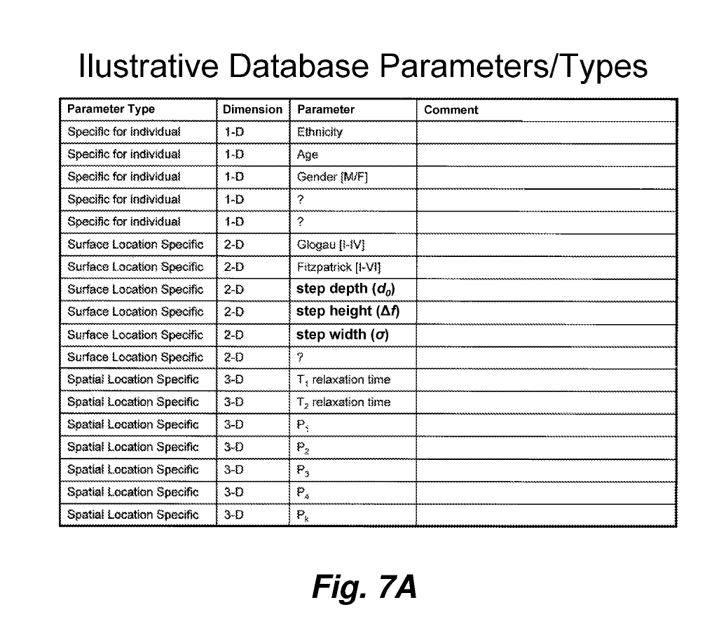

Turning now to FIGS. 7A and 7B, a block diagram is shown of a data model 400 for a skin type database 244 in accordance with one embodiment of the present invention. As shown, this model 400 of data organization within the database 244 includes tables having as their primary keys (“pk”) various pieces of data particularly relevant to a database of skin type information. In addition, those tables which have a many-to-one relationship to one or more other tables also include primary key information (designated as foreign keys (“fk”)) for those related tables.

The data model can be organized as a “flat file” data structure or, in certain embodiments, can comprise a relational data structure. Thus, for example, a single subject can give rise to multiple skin type measurements. Each skin type measurement can give rise to multiplicity of measured parameters. In such instance, there can be a one to many relationship between the subject and the samples, and between the samples and the measured parameters. Particular, where the data is annotated, e.g., by reference to a foreign (e.g., patient record) database, linkage may be provided by various foreign keys (fk). The relationships between the entities may be optional or mandatory.

Various parameters that can be included in skin-type database records include, but are not limited to one or more of the following NMR or other quantitative measurement parameters, values derived/calculated from the quantitative parameters (skin-type values) Glogau scale value(s), Fitzpatrick scale value(s), subject identifier, gender identifier, age identifier, ethnicity identifier, location of the measurement(s), notes or comments, and the like.

### IV. Graphical User Interface for Skin Type Database.

In certain embodiments the invention is provided together with a suite of functions made available to users through a collection of user interface screens (e.g., HTML pages). Typically, the interface will have a main menu page from which various options can be selected.

For example, the main menu can provide options for imputing quantitative and/or qualitative skin measurement data (e.g., manually entered, read from a computer readable media, or input from a network link) for updating and/or querying a skin type database. Other options can be provided for annotating data in the skin type database, for importing ancillary medical record information, for exporting information to a medical record database, and the like.

Preferably, the user interface employed with this invention possesses similar attributes to interfaces for other medical and/or research databases. Examples of other databases including similar interfaces might include interfaces for users such as a hospital records department, a physician, an insurance provider, and the like. In certain embodiments the “look and feel” of each of these databases will resemble one another. For example, each might contain a commonly formatted collection of query buttons output formats, treatment summaries, and the like. As a result the system may bring one of multiple available “query” screens, each commonly formatted to allow the user to formulate his or her query. Upon execution of this query, the system may present an appropriate results screen (again of common format) presenting the results of the executed query.

By providing these features as a common interface spanning multiple databases, users familiar with one database interface can quickly learn to navigate through related databases. Thus, they will be able to leverage their knowledge of formulating appropriate queries and locating desired skin characterization and/or treatment plan information obtained from working with an initial database.

### V. Treatment: Methods, Systems, Devices.

In the past, grading scales for skin type/character were devised to assess clinical outcomes from ablative, and other technologies, grouping the various aspects of skin damage and/or aging into broad but useful classification schemes. The most widely used include the Glogau and Fitzpatrick wrinkle assessment scales. These well-accepted grading scales were primarily developed and used in evaluating ablative, and other technologies, such as chemical peeling or carbon dioxide laser resurfacing, which result in improvement in all aspects of skin damage and/or aging. On the other hand, these scales were not intended to individually or independently assess each of the diverse aspects of the aging skin, but rather to group findings together into stages of progression.

Since those scales were devised, nonablative technologies have emerged and rapidly evolved in an effort to minimize risk and speed recovery in the face of acceptable cosmetic improvement. For example, nonablative laser resurfacing technologies typically target specific aspects of skin damage and/or aging but not all, making broad groupings of clinical findings less useful in assessing their efficacy. In addition, patients seeking nonablative treatments often do not fall neatly into any one global category, displaying certain aspects of skin aging but not others. Separating the various facets of skin aging and/or damage from each other, e.g., by querying the skin type database(s) as described herein, permits the quantitative assessment of nonablative and other modalities that target individual aspects of the aging skin.

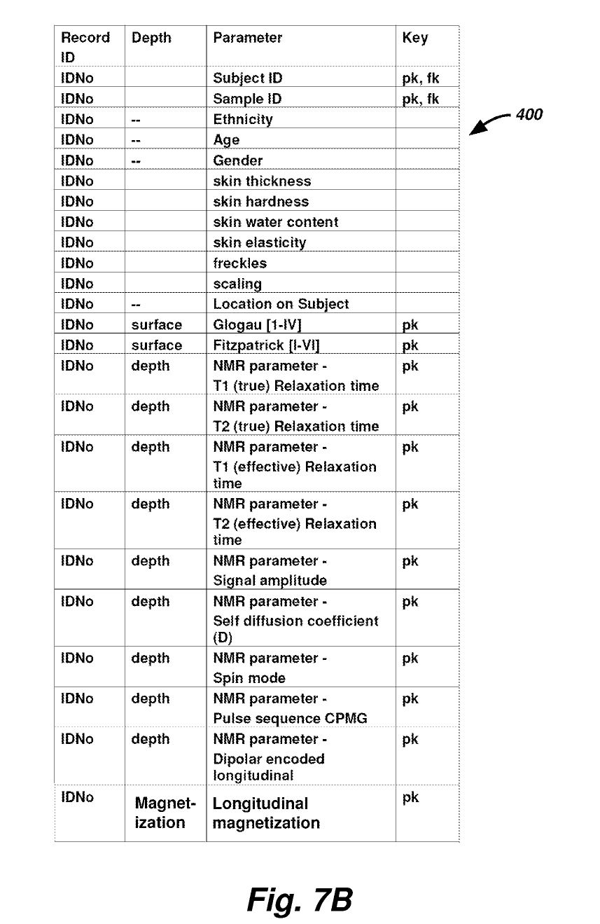

Accordingly, in certain embodiments, this invention provides systems for the treatment of subjects where the systems utilize one or more quantitative measures of skin type (NMR, MRI, PET, thermography, capacitance, resistance, etc.) alone or in conjunction with a skin type database as described herein. In various embodiments the systems include one or more processors configured to receive quantitative measurement(s) from the patient/subject and, optionally, to calculate derived values from such measurement(s), optionally a database storing a library of skin type (e.g., NMR) characterizations (i.e., a skin type database); and a processor coupled to the database to access the library of skin type characteristics and, optionally, to generate a treatment plan optimized for a patient skin type characterized by the measured quantitative (e.g., NMR) parameters. FIG. 8 shows a block diagram illustrating one embodiment of such a system.

As shown in FIG. 8, in this instance, the system comprises a patient treatment system 500, for obtaining diagnostic data (e.g., NMR data), generating a skin characterization and/or treatment plan, and outputting the treatment plan and/or delivering the treatment to the patient. As illustrated in FIG. 8, the system 500 can include a diagnostic system or module 510, optionally a treatment planning system 520, and, optionally, a treatment output system 540. In certain embodiments the diagnostic system or module 510, treatment planning system 520, and, when present, treatment output system 540 can all be at the same location. Alternatively they can be used in different locations and/or at different times. Thus, for example the diagnostic information can be obtained at a different time and/or location and later provided to the treatment planning and/or treatment delivery system. In certain embodiments the diagnostic system or module 510, treatment planning system 520, and, when present, treatment output system 540 are all incorporated into a single treatment device. In certain embodiments the diagnostic system or module 510, and treatment planning system 520, are combined into a single device, in certain embodiments the treatment planning system 520 and treatment output system 540 are incorporated into a single device, and in certain embodiments, the diagnostic system or module 510 and the treatment output system 540 are incorporated into a single device.

The diagnostic system or module typically comprises a means of acquiring quantitative measures (e.g., NMR data, x-ray data, PET data, thermograhic data, etc.) of the skin to be treated. As illustrated the diagnostic system comprises an NMR detector 502 to acquire NMR measures of skin properties. The detector is, optionally, operatively coupled to a digital processing system to facilitate processing of the acquired data and/or communication of acquired data or processed data to a treatment planning system. In certain embodiments, however, the acquired data could simply be manually re-entered into the treatment planning system and/or the treatment delivery system or transferred via a removable/portable storage system (e.g. a CD, a flash memory, a portable hard drive, etc.). As indicated above, the diagnostic system/module 510 can include any system capable of producing quantitative information regarding skin properties in a patient that may be used for subsequent medical diagnosis, treatment planning and/or treatment delivery. For example, diagnostic imaging system 510 can comprise an NMR detector 502 as illustrated, and/or a computed tomography (“CT”) system, a magnetic resonance imaging (“MRI”) system, a positron emission tomography (“PET”) system, an ultrasound system, a thermographic system, and/or the like. For ease of discussion, diagnostic imaging system 500 may be discussed below at times in relation to an NMR modality.

In certain embodiments, the diagnostic system 510 comprises an NMR detector 502 which can be coupled to a digital processing system 504 to control the NMR measurement and to process NMR data (e.g., to provide the Fourier transformation of the raw data, to calculate derived skin-type values, etc.). The diagnostic system 510 can include a bus or other means 506 for transferring data and commands to and from the NMR detector 502 and/or the treatment planning system 520 and/or the treatment delivery system 540. Digital processing system 504 can include one or more general-purpose processors (e.g., a microprocessor), special purpose processor such as a digital signal processor (“DSP”) or other type of device such as a controller or field programmable gate array (“FPGA”). Digital processing system 502 can also include other components (not shown) such as memory, storage devices, network adapters and the like. Digital processing system 502 can transmit diagnostic data (e.g., NMR data) to treatment planning system 520 over a data link 506, which can be, for example, a direct link, a wireless link, a local area network (“LAN”) link or a wide area network (“WAN”) link such as the Internet. In addition, the information transferred between systems may either be pulled or pushed across the communication medium connecting the systems, such as in a remote diagnosis or treatment planning configuration. In remote diagnosis or treatment planning, a user may utilize embodiments of the present invention to diagnose or treatment plan despite the existence of a physical separation between the system user and the patient.

In certain embodiments treatment planning system 520 includes a processing device 526 to receive and process quantitative skin data (e.g., NMR data). Processing device 526 can represent one or more general-purpose processors (e.g., a microprocessor), special purpose processor such as a DSP or other type of device such as a controller or FPGA. Processing device 526 can be configured to execute instructions for performing treatment planning operations discussed herein.

In various embodiments treatment planning system 520 can also include system memory 522 that may include a random access memory (“RAM”), or other dynamic storage devices, coupled to processing device 526 by bus 532, for storing information and instructions to be executed by processing device 526. System memory 522 also can be used for storing temporary variables or other intermediate information during execution of instructions by processing device 526. System memory 522 can also include a read-only memory (“ROM”) and/or other static storage device coupled to bus 532 for storing static information and instructions for processing device 526.

Treatment planning system 520 can also include storage 524, representing one or more storage devices (e.g., a magnetic disk drive or optical disk drive) coupled to bus 532 for storing information and instructions. Storage device 524 can be used for storing instructions for performing the treatment planning steps discussed herein. Thus, for example, in certain embodiments, storage 524 can comprise a machine-accessible medium that provides instructions that, if executed by a machine, will cause the machine to perform operations comprising: receiving nuclear magnetic resonance (NMR) data; querying an NMR skin type database to identify the skin type characterized by said NMR data; calculating and outputting to a display, tangible medium, and/or treatment device a treatment plan optimized for the skin type characterization returned from the query.

Processing device 526 may also be coupled to a display device 528, such as a cathode ray tube (“CRT”) or liquid crystal display (“LCD”), for displaying information to the user. An input device 530, such as a keyboard, and/or mouse and the like, can be coupled to processing device 526 for communicating information and/or command selections to processing device 526. One or more other user input devices (e.g., a mouse, a trackball or cursor direction keys) may also be used to communicate directional information, to select commands for processing device 526 and to control cursor movements on display 528.

In various embodiments the processing device 526 will be configured to query or to hand a query to a skin type database 244 as described herein. It will be recognized that the skin type database 244 can be a local component of system 500, or it can be remote.

It will be appreciated that treatment planning system 520 represents only one example of a treatment planning system, that can have many different configurations and architectures, that can include more components or fewer components than treatment planning system 520 and that can be employed with the present invention. For example, some systems often have multiple buses, such as a peripheral bus, a dedicated cache bus, etc.

In various embodiments treatment planning system 520 may share its database (e.g., data stored in storage device 524 and/or data or treatment plans calculated and/or returned from a query to skin type database 244) with a treatment delivery system 540, comprising, for example, a radiation treatment delivery system 542, so that it may not be necessary to export from the treatment planning system prior to treatment delivery. In various embodiments treatment planning system 520 can be linked to treatment delivery system 540 100 via a data link 534, that can be a direct link, a wireless link, a LAN link or a WAN link as discussed above with respect to data link 506. It should be noted that when data links 506 and 534 are implemented as LAN or WAN connections, any of diagnostic system 510, treatment planning system 520 and/or treatment delivery system 540 can be in decentralized locations such that the systems may be physically remote from each other. Alternatively, any of diagnostic imaging system of diagnostic system 510, treatment planning system 520 and/or treatment delivery system 540 can be integrated with each other in one or more systems or even in a single device.

In certain embodiments treatment delivery system 540 can include a therapeutic and/or surgical radiation source 542 (e.g., a laser) to administer a prescribed radiation dose to a target in conformance with a treatment plan. In various embodiments the treatment delivery system 540 can optionally an imaging system 544 (including imaging sources and detectors) to capture intra-treatment images of a treatment site for registration or correlation with the diagnostic information described above in order to position the patient with respect to the radiation source. In various embodiments treatment delivery system 540 can optionally include a digital processing system 546 to control therapeutic radiation source 542 (treatment device) and/or imaging system 544, and/or, optionally, a patient support device such as a treatment couch. Digital processing system 546 can include one or more general-purpose processors (e.g., a microprocessor), special purpose processor such as a DSP or other type of device such as a controller or FPGA. Digital processing system 546 can also include other components (not shown) such as memory, storage devices, network adapters and the like. In various embodiments digital processing system 546 can be coupled to treatment device (e.g., radiation source 542), imaging system 544 and treatment by a bus 548 or other type of control and communication interface.

Also, in various embodiments, this invention provides methods of treating a subject. In various embodiments these methods typically involve making one or more quantitative measurements (e.g., NMR measurements) of one or more regions of skin on the subject. Calculated/derived metrics (e.g., skin type values) can be optionally calculated from the quantitative parameters measured. In certain embodiments the derived metrics are used directly in characterizing the skin and/or planning a treatment or the measurements and/or derived data are used to a skin-type database and to thereby identify/characterize the skin type of the subject. In various embodiments the calculated data, and/or derived skin type characterization that can be delivered to a display or printer and/or to a treatment system, and/or stored to a computer readable medium.

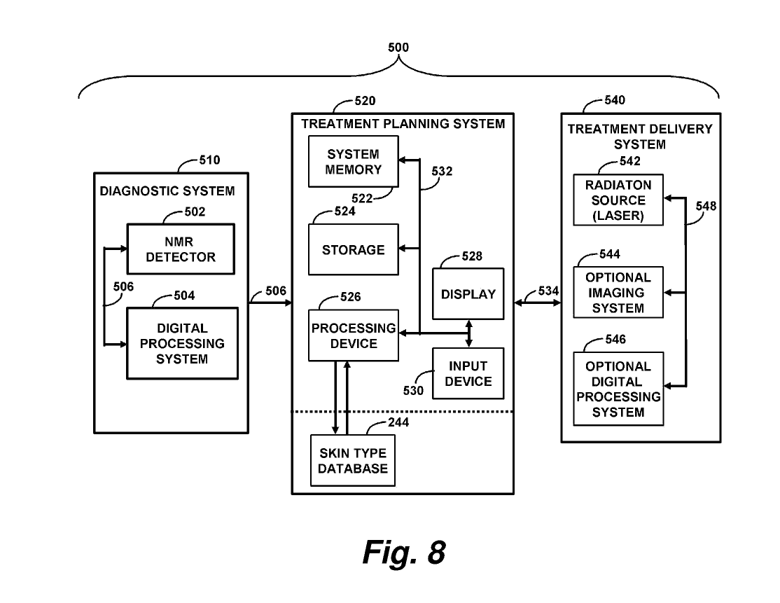

One such method is schematically illustrated in FIG. 9. As illustrated in process 600 in FIG. 9 patient data (e.g., NMR data) can be provided from any of a number of sources including, but not limited to a computer terminal 610, a network link 612, an internet connection 612, a local network 614 (e.g., via an intranet, or over a bus on a local system), or from a scanning/treatment system 616 (e.g., from a diagnostic system/module 510 therein), and the like. The patient data can, optionally processed, 619 to calculate derived values (e.g., skin-type values). The patient data and/or derived values can be used to query a skin type database 244 as shown in step 622. This process can optionally involve querying a medical record database and such query can be handled as part of the initial query and/or as a subsequent query produced by the skin type database system. The query results are returned thereby identifying a skin type as illustrated in step 624 which, in various embodiments, can be delivered, e.g., as a skin type classification and/or treatment plan, as shown in step 626. The results can optionally be inspected, and altered, and/or annotated to produce as illustrated in step 622 to provide a revised query to the skin type database to further optimize the skin type characterization and/or treatment plan. The skin type classification can ultimately be delivered to any convenient output device 628 (e.g., computer monitor, computer readable media, network connection, and the like) and/or to a treatment delivery system 540.

It will be appreciated that treatment method shown in FIG. 9 represents only one example of a treatment method, that can have many different configurations and can include more steps or fewer steps than shown in process 600 and that can be employed as described herein.

### EXAMPLES

The following examples are offered to illustrate, but not to limit the claimed invention.

### Example 1

### Statistical Study of Facial Skin by Mobile NMR

### Summary

Portable one-sided NMR/MRI can be used for non-invasive characterization of skin without the need for huge, expensive and immobile clinical MRI scanners. Furthermore, higher spatial resolution through the skin is available with the modified mouse because of the inherently strong magnetic field gradients of the mobile device. A specially designed NMR mouse was used to study the facial skin of 43 female adults of different age and skin color. Relaxation weighted depth profiles were measured in the lower half of the face covering 3 mm depths and statistically analyzed for correlations with the Glogau and Fitzpatrick scales. High moisture content of the cutis determined by NMR was found to correlate with younger age and darker skin color. Additionally, this work lays the foundation for the characterization of the administration and dissipation of filler which can be followed non-invasively and quantitatively using the protocol(s) described herein.

### Introduction

Magnetic resonance imaging (MRI) [1, 2] explores the resonance of atomic nuclei in a magnetic field with radio-frequency irradiation by the phenomenon of Nuclear Magnetic Resonance (NMR) [3] to generate tissue-specific contrast in images of living species [4] and dead matter [5, 6]. Images detailing the stratigraphy of skin are hard to obtain by conventional MRI machines, and special coils [7,10] or dedicated scanners are needed [11-17]. Yet skin is the largest organ of humans, covering about two square meters in area. It is the interface of the body to the environment, which protects the body and plays a defining role in the perceived identity of individuals. For optimum skin care and medical treatment, unambiguous skin typing is essential. So far skin characterization is subject to the varying qualifications of the skin specialist. Established scales for skin typing are the Glogau wrinkle scale [18] and the Fitzpatrick color scale [19].

The move from the somewhat subjective scales to an objective scale requires a measurement device which gives the same results in the hands of different operators. While MRI could serve that purpose, conventional machines are far too bulky and expensive, and they lack the necessary high spatial resolution through the depth of the skin. Portable MRI machines like the Profile NMR-MOUSE [20] have emerged that measure the information of one pixel in a medical image locally with low lateral resolution of about 10 mm×19 mm but depth resolution of better than 5 μm. They are small, affordable, and first studies have revealed, that different skin layers can be resolved and inter-individual variations be observed [16]. Envisioning, that skin can be mapped in this way across the whole body with different contrast parameters, we conducted a pilot statistical study to investigate the applicability and reliability of this approach to the in vivo study of skin, and the correlation of parameters retrieved from the NMR measurements with the Glogau and Fitzpatrick scales.

### EXPERIMENTAL

### Profile NMR-MOUSE

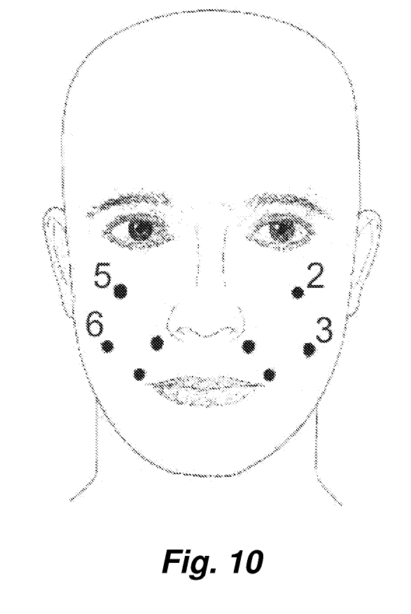

The measurements were conducted with the Profile NMR MOUSE®. This is a purse-size NMR device capable of acquiring NMR signals from a flat slice at a given distance above the surface of the sensor (FIG. 11). For the sensor used, the slice was located 5 mm above the sensor. Its lateral extensions were about 1 cm², and the slice thickness was 50 μm. The sensor was mounted on a step-motor driven precision lift by means of which the distance of the sensor surface to the skin was adjusted, shifting the sensitive slice through the skin step by step to scan a depth profile (FIG. 11).

TABLE 3
Experimental parameters for NMR depth profiling of skin.

| Parameter | Value | |
|---|---|---|
| recycle delay | 0.5 | s |
| number of cans | 8 | |
| NMR frequency | 17.1 | MHz |
| duration of the 90° pulse | 5 | μs |
| duration of the 180° pulse | 5 | μs |
| echo time | 0.06 | ms |
| number of echoes per scan | 500 | |
| acquisition time per echo | 0.02 | ms |

Each profile covered a depth of 2.5 mm with 50 points spaced equally apart every 50 μm. At each position a CPMG multi-echo train [21, 22] was measured. The lift with the sensor was mounted underneath an examination table to provide the volunteer with some comfort during the time of about 5 minutes needed for the measurement of one profile. Each point of the depth profile was calculated from the amplitudes of the 400 echoes acquired by adding the amplitudes of the first 64 echoes and normalizing this sum to the sum of the remaining echo amplitudes. In the resultant value of the profile, this introduces a weight of the transverse relaxation time T₂ to the signal amplitude. The measurement parameters (Table 3) such as the recycle delay, the number of scans, the durations of the radio-frequency excitation pulses, the echo time, the number of echoes per scan, and the acquisition time per echo as well as the summation parameters for reduction of the echo train to one number in the profile were optimized for maximum contrast and speed of measurement.

Subjects:

Forty three female volunteers without pathological findings were investigated, and high-resolution NMR depth profiles measured at six cheek positions, three on each side, as shown in FIG. 10. The female population covered large ranges of the Glogau and Fitzpatrick scales, and the corresponding NMR data were analyzed statistically.

Evaluation of Skin Profiles:

The experimental skin depth profiles p(d) were analyzed by eyeball fits with a convolution of a heaviside step function f(d) and a Gauss function g(d, σ), where d is the depth parameter and σ is the standard deviation of the Gauss function:

p(d)=f(d) [symbol] g(d,σ). (1)

Three parameters were extracted from the fit: the position d₀ of the step, the standard deviation σ defining the width of the step, and the step height Δf. These parameters were interpreted in terms of physical parameters known to affect T2 relaxation and further analyzed for correlations with the Glogau and Fitzpatrick ratings of the subject's skin.

The distribution of the female subjects according to the Glogau-Fitzpatrick matrix is reported in Table 4. Not all skin types are represented. In particular, the populations of the Fitzpatrick 5 and 6 ratings are too low for a statistical analysis. The same applies to the number of male volunteers. This is why these measurement results are not reported.

TABLE 4
Statistics of measured female volunteers according to the Glogau and
Fitzpatrick scales

| Female | Glogau 1 | Glogau 2 | Glogau 3 | Glogau 4 |
|---|---|---|---|---|
| Fitzpatrick 1 | 1 | 0 | 0 | 0 |
| Fitzpatrick 2 | 3 | 6 | 5 | 5 |
| Fitzpatrick 3 | 3 | 4 | 2 | 0 |
| Fitzpatrick 4 | 4 | 4 | 2 | 2 |
| Fitzpatrick 5 | 1 | 1 | 0 | 0 |
| Fitzpatrick 6 | 0 | 0 | 0 | 0 |

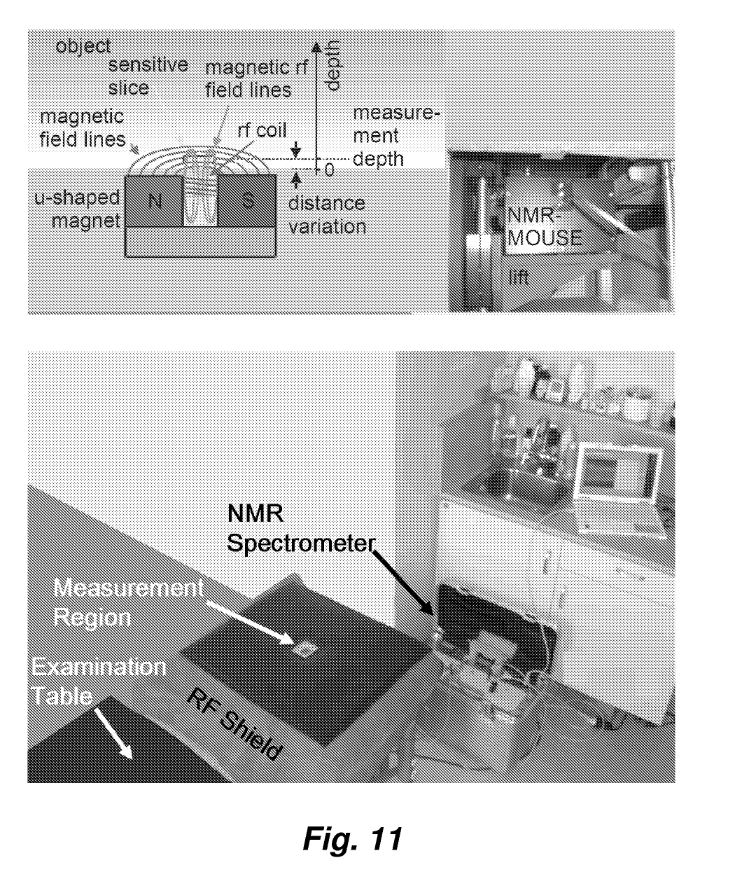

Representative skin depth profiles of facial points 1 to 7 (cf FIG. 10) of one subject are plotted in FIG. 12. The profile amplitude starts at a lower value near the surface of the skin and changes to a higher value between 1 and 1.5 mm depth. Higher amplitude denotes a higher number of protons in mobile molecules. Low amplitudes are characteristic of a lower number of protons or protons in less mobile molecules. The low amplitudes are found in the outer 1 mm of the skin which is identified as the cutis. The higher amplitudes correspond to the subcutis. At sufficiently high depth resolution, the strata of the cutis can be resolved as well [16].

The statistical evaluation of the skin profiles was restricted to the profiles of the cheek, and for further improvement of the signal-to-noise ratio, the cheek profiles were averaged for each volunteer. If only one useful profile of the cheek was available, this profile was discarded.

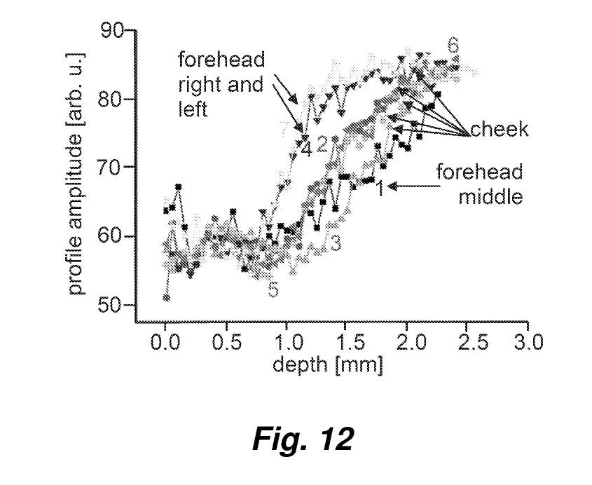

This reduction of the experimental data produced one average curve for the depth profile of the facial skin per volunteer. This curve was fitted with the expression (1) and the fit parameter position d₀ of the step, step width σ, and the step height Δf determined (FIG. 13A). The parameter spread was separated into 5 bins as demonstrated in FIG. 13B for the distribution of the step height Δf and the entries were analyzed for their distributions according to Glogau and Fitzpatrick type.

If P(x_{G}) is the normalized distribution of volunteers according to the Glogau scale x_{G}=1, . . . , 4, P(x_{F}) the normalized distribution of volunteers according to the Fitzpatrick scale x_{F}=1, . . . , 6, and P(pᵢ) the distribution of fit parameters pᵢ=d₀i, σᵢ, Δfᵢ over the bins i, joint probability densities W(x_{G}, pᵢ) and W(x_{F}, pᵢ) can be defined such that:

P(x_{G})=W(x_{G},pᵢ)P(pᵢ) and P(x_{F})=W(x_{F},pᵢ)P(pᵢ) (2)

These joint probability densities are found by analyzing the entries in each bin for their distributions according to the Glogau or Fitzpatrick scales. They reveal correlations of the fit parameters with the Glogau and Fitzpatrick ratings. The 20 distributions W(x_{G}, pᵢ) and W(x_{F}, pᵢ) extracted from the fit parameters are displayed in FIG. 7. As for such a statistical analysis the number of volunteers investigated is small, the uncertainties in the distributions are high, and only trends can be identified (gray arrows).

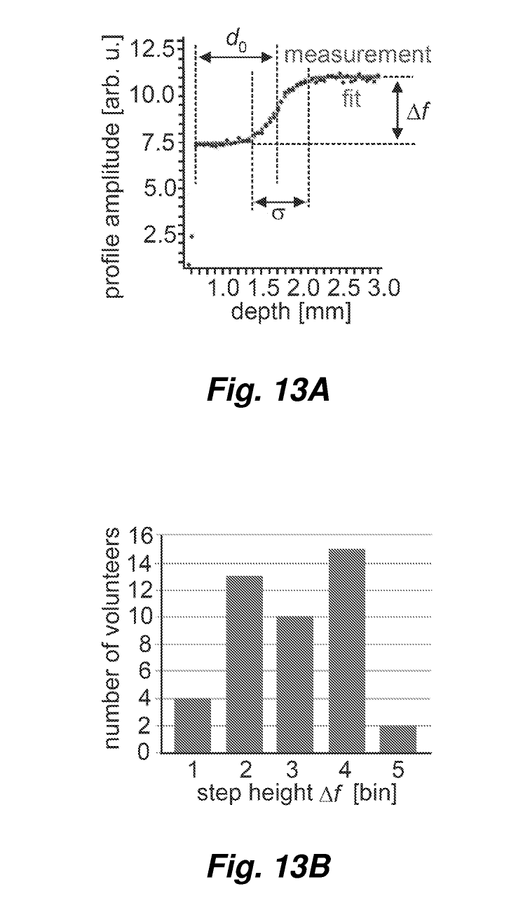

The most pronounced correlations are found for the step height Δf (FIG. 14, top). Small step heights correlate with small Glogau ratings and high Fitzpatrick ratings. By interpreting the signal amplitudes in terms of moisture content, small step heights mean small differences in moisture content between cutis and subcutis and thus a comparatively well moisturized cutis. Our results show that high moisture content of the skin is observed for young and dark skin. Correlations with the step position (FIG. 14, middle) which measures the combined thickness of the epidermis and cutis and capillary dermis (including any denatured proteis) higher d₀ is also observed for higher F values. The step width (FIG. 14, bottom) is most difficult to determine, as any deviation from a parallel alignment of the sensor surface with the skin leads to an apparent widening of the step width. Our data do however reveal correlations and allow conclusions regarding sigma vis a vis Glogau and/or Fitzpatrick scale values. The significance for dermatological investigation can not be underestimated since this method shows the ability to make precise structural and pathophysiological measurements of skin in vivo without resorting to traditional incisional biopsy and alteration of the skin itself. Further study has demonstrated the utility of using these methods to investigate therapies as diverse as topically applied pharmacologic agents and intra-dermal and sub-dermal injectable medical device implants.

TABLE 5
Subcutaneous fillers and their properties.

| | Filler and | | | T_{2eff} [ms] at |
|---|---|---|---|---|
| Product Name | concentration | Buffer | T₁ [ms] | t_{E} = 60 μs |
| Juwederm | hyaluronic acid, | phosphate | 1940 ± 29 | 4.8 ± 0.6 |
| ultra | 24 mg/ml | buffer | | 41.2 ± 0.1 |
| Perlane | hyaluronic acid, | phosphate | 2118 ± 65 | 11.3 ± 1.8 |
| | 24 mg/ml | buffer | | 44.1 ± 0.4 |
| Restylane | hyaluronic acid, | phosphate | 2146 ± 49 | 8.0 ± 1.0 |
| 4 ml | 24 mg/ml | buffer | | 45.3 ± 0.3 |
| Restylane | hyaluronic acid, | NaCl | 1947 ± 47 | 8.5 ± 1.1 |
| lipp | 24 mg/ml | solution | | 44.9 ± 0.3 |
| Cosmoderm | human-based | NaCl | 1320 ± 24 | 6.2 ± 1.1 |
| 1 | collagen | solution + | | 43.3 ± 0.2 |
| | 34 mg/ml | Lidocain | | |
| | | 0.3% | | |

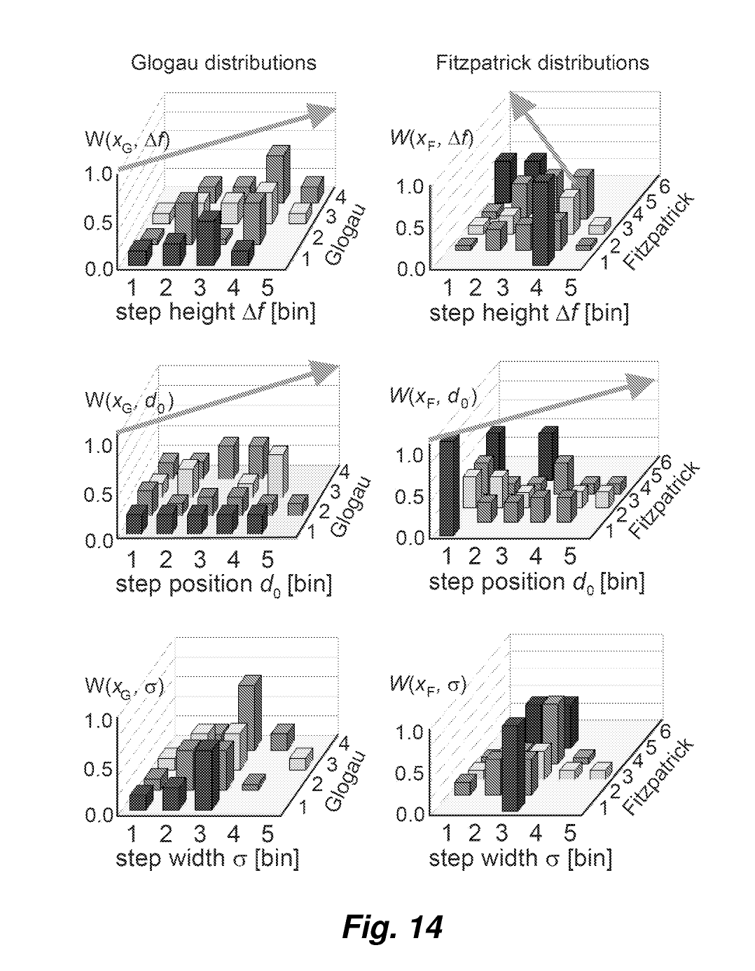

In another set of measurements, the NMR-MOUSE was tested for its use in detecting the presence of subcutaneous fillers and discriminating their types. The investigated fillers, their concentrations, buffer additive, longitudinal NMR relaxation times T₁, and transverse NMR relaxation times T₂ are listed in Table 5. As the magnetization decay is bi-exponential, two values of T₂ were obtained from a biexponential fit. The data were acquired with the CPMG sequence using the same NMR-MOUSE as for the skin depth profiles. The transverse relaxation time T₂ is an effective relaxation time T_{2eff} and the echo time t_{E} of acquisition pulse sequence needs to be specified (cf. Table 5) as the NMR-MOUSE employs an inhomogeneous magnetic field with a gradient of about 20 T/m. It turns out, that the investigated products can all be distinguished in their pure forms based on their NMR relaxation times T₁ and T_{2eff}. This is illustrated in FIG. 15 in graphical form.

Two fillers were injected underneath the skin of the arm of a volunteer, Cosmoderm and Restylane. Both fillers lower the signal amplitude of the subcutis (FIG. 15). Moreover, the signal of pure Restylane is lower than that of pure Cosmoderm, and this difference also shows up after injection into the arm: the subcutus with Restylane shows a lower signal than the subcutis with Restylane. Interestingly, the Restylane profile reveals, that Restylane was injected nearly one millimeter deeper than Cosmoderm, as part of the subcutis signal without filler can be identified between 1.0 and 1.5 mm depth. As the difference between the depth profiles before and after filler injection is considerable, the effect of filler treatment can be followed with the NMR-MOUSE. In particular, the depth of injection can be quantified, and the resorption can be followed and quantified.

### SUMMARY AND DISCUSSION

The facial skin of a statistically relevant number of female volunteers was analyzed in terms of depth profiles with the NMR-MOUSE. The most pronounced feature of the depth profiles is a step at the interface of cutis and subcutis. The experimental depth profiles were fitted with a model function, and the fit parameters depth of the cutis, step height, and step width extracted. These fit parameters were subsequently analyzed in terms of joint probability densities to identify correlations with the Glogau and Fitzpatrick ratings of the skin types. A clear correlation was found with the step height, which reports about the difference in moisture content between cutis and subcutis. Young and dark skin exhibits a larger step height than older and fairer skin. The NMR skin-depth profiling technology can also be used to quantify the effect of filler treatments, and different fillers can be distinguished.

This is the first extensive study of skin with a portable NMR device. Several shortcomings in the device and measurement procedure became evident during the investigation. In particular the patient comfort during the measurement needs to be improved and the measurement time to be reduced. The former can be achieved by a construction of a suitable positioning device. The latter can be solved with the construction of a high-resolution NMR depth profiler which measures the Fourier transform of the depth profile [20, 23]. The depth profile is then obtained by Fourier transformation of the measured signal. This will reduce the measurement time from several minutes to below one minute. Such a skin NMR sensor is currently under construction.

The results obtained in this study together with the identified improvements encourage the development of an NMR skin mapping methodology by which the skin of individuals can be mapped and characterized in comparison with reference skin maps. The sensitivity of the NMR depth profile can be adapted to different parameters in a manner similar to setting the contrast in medical MRI. NMR skin maps can then be used to identify skin treatment procedures and quantify the success of such procedures.

### REFERENCES

[1] Lauterbur et al. (1973) Nature 242: 190-191.
[2] Mansfield and Grannell (1973) J. Phys. C 6: L422.
[3] Bodenhausen and Wokaun (1987) Principles of Nuclear Magnetic Resonance in One and Two Dimensions, Clarendon Press, Oxford.
[4] Haake et al. (1999) Magnetic Resonance Imaging, Wiley-Liss, New York.
[5] P. T. Callaghan, Principles of Nuclear Magnetic Resonance Microscopy, Clarendon Press, Oxford, 1991.
[6] B. Bluemich, NMR Imaging of Materials, Clarendon Press, Oxford, 2000
[7] S. Richard, B. Querleux, J. Bittoun, I. Idy-Peretti, O. Jolivet, E. Cermakova, J.-L. Leveque, In vivo proton relaxation time analysis of the skin layers by magnetic resonance imaging, J. Investig. Derm. 97 (1991) 120-125.
[8] S. Richard, B. Querleux, J. Bittoun, O. Jolivet, I. Idy-Peretti, O. de Lacharriere, J.-L. Le'veAque, Characterization of the skin in vivo by high resolution magnetic resonance imaging: water behavior and age-related effects, J. Investig. Derm. 100 (1993) 705-709.
[9] B. Querleux, S. Richard, J. Bittoun, O. Joliveto, I. Idy-peretti; R. Bazin, J. L. Leveque, In-vivo hydration profile in skin layers by high-resolution magnetic resonance imaging, Skin Pharmacol. 7 (1994) 210-216.
[10] F. Mirrashed, J. C. Sharp, In vivo quantitative analysis of the effect of hydration (immersion and Vaseline treatment) in skin layers using high-resolution MRI and magnetization transfer contrast, Skin Res. Tech. 10 (2004) 14-22.
[11] G. Bennett, J.-P. Gorce, J. L. Keddie, P. J. McDonald, H. Bergling, Magnetic resonance profiling studies of the drying of film-forming aqueous dispersions and glue layers, Magn. Reson. Imaging 21 (2003) 235-241.
[12] Dias et al. (2003) J. Phys. D: Appl. Phys. 36: 364-368.
[13] L. Backhouse, M. Dias, J. P. Gorce, K. Hadgraft, P. J. McDonald, J. W. Wiechers, GARField magnetic resonance profiling of the ingress of model skin-care product ingredients into human skin in vitro, J. Pharm. Sci. 93 (2004) 22742283.
[14] P. J. McDonald, A. Akhmerov, L. J. Backhouse, S. Pitts, Magnetic Resonance Profiling of Human Skin In Vivo Using GARField Magnets, J. Pharmac. Res. 94 (2005) 1850-1860.
[15] P. Doughty, P. J. McDonald, Drying of coatings and other applications with GARField, in: S. Stapf, S. Han (Eds.), NMR in: Chemical Engineering, WileyVCH, Weinheim, 2006, pp. 89-107.
[16] F. Casanova, J. Perlo, B. Bluemich, Depth profiling by single-sided NMR, in: S. Stapf, S. Han (Eds.), NMR in Chemical Engineering, Wiley-VCH, Weinheim, 2006, pp. 107-123.
[17] B. Blümich, J. Perlo, F. Casanova, Mobile single-sided NMR, Prog. Nucl. Magn. Reson. Spectr. 52 (2008) 197-269.
[18] R. G. Glogau, Aesthetic and anatomic analysis of the aging skin, Semin. Cutan. Med. Surg. 15 (1996) 134-138.
[19] T. B. Fitzpatrick, Solei et peau, J. Med. Esthet. 2 (1975) 33-34.
[20] J. Perlo, F. Casanova, B. Bluemich, Profiles with microscopic resolution by single-sided NMR, J. Magn. Reson. 176 (2005) 64-70.
[21] H. Y. Carr, E. M. Purcell, Effects of diffusion on free precession in nuclear magnetic resonance experiments, Phys. Rev. 94 (1954) 630-638.
[22] S. Meiboom, D. Gill, Modified spin-echo method for measuring nuclear relaxation times, Rev. Sci. Instrum. 29 (1958) 688-691.
[23] W.-H. Chang, J.-H. Chen, L.-P. Hwang, Single-sided mobile NMR with a Halbach magnet, Magn. Reson. Imaging 24 (2006) 1095-1102.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of characterizing skin type in a human subject, said method comprising:
performing nuclear magnetic resonance (NMR) measurements at a plurality of skin depths at one or more locations on said subject; and
calculating from data provided by said NMR measurements one or more skin-type values indicative of a skin characteristic.

2. The method of claim 1, wherein said skin-type values are indicative of a Glogau and/or a Fitzpatrick scale value.

3. The method of claim 1, wherein said NMR measurements are made using a portable NMR device.

4. The method of claim 1, wherein said calculating comprises determining a Fourier transform of an NMR signal depth profile.

5-7. (canceled)

8. The method of claim 1, wherein said calculating comprises determining an NMR signal amplitude as a function of skin depth at a skin location.

9. The method of claim 8, wherein said calculating comprises determining an NMR signal amplitude as a function of skin depth wherein said signal amplitude as a function of skin depth defines a step.

10. The method of claim 8, wherein said calculating comprises identifying a step depth (d₀), optionally identifying a step height (Δf), and optionally identifying a step width (σ).

11. The method of claim 8, wherein said skin-type values are a function of a step depth (d₀) and/or a step height (Δf), and/or a step width (σ).

12. The method of claim 8, wherein said calculating comprises determining a location of a transition between cutis and subcutis and/or comprises determining a thickness of cutis and/or subcutis.

13. (canceled)

14. The method of claim 1, wherein said NMR measurements comprise measurements of one or more parameters selected from the group consisting of: relaxation time T2 (true), relaxation time T2 (effective), relaxation time T1 (true), relaxation time T1 (effective), self-diffusion coefficient D, signal amplitude, spin modes, pulse sequence CPMG, dipolar encoded longitudinal magnetization, multiquantum build-up, multiquantum decay, diffusion coefficients, chemical shift resolved spectra, component amplitudes in chemical shift resolved spectra, and/or a mathematical function thereof.

15. The method of claim 1, further comprising outputting to a patient medical record said one or more NMR measurements and/or said one or more skin-type values.

16. The method of claim 1, further comprising outputting to a display or printer and/or storing to a computer readable medium said one or more NMR measurements and/or said one or more skin-type values.

17. The method of claim 15, wherein said patient medical records comprise one or more of the following: Glogau value for the same skin, Fitzpatrick value for the same skin, skin thickness, skin hardness, skin water content, freckles, scaling, subject identifier, subject age, subject ethnicity, subject gender, and location of skin measurement.

18. A method of identifying a skin type for a region of skin of a treatment subject, said method comprising:
providing a skin type database containing skin type records from a plurality of subjects;
receiving one or NMR parameters determined from said region of skin and/or skin-type values calculated from said NMR parameters;
querying said skin type database using said one or more NMR parameters and/or skin-type values to identify the skin type of said subject; and
outputting to a display or printer and/or storing to a computer readable medium a characterization of the skin type for said region of skin of said subject.

19. The method of claim 18, wherein said skin-type values are indicative of a Glogau and/or Fitzpatrick scale value.

20. (canceled)

21. The method of claim 18, wherein said NMR parameters are determined using a portable NMR device.

22. The method of claim 18, wherein said receiving comprises calculating or receiving already a calculated Fourier transform of an NMR signal depth profile.

23-25. (canceled)

26. The method of claim 18, wherein said receiving comprises receiving a measurement of an NMR signal amplitude as a function of skin depth at a location skin location.

27. The method of claim 26, wherein said receiving comprises receiving an NMR signal amplitude as a function of skin depth wherein said signal amplitude as a function of skin depth defines a step.

28. The method of claim 26, wherein said receiving comprises calculating or receiving already calculated a step depth (d₀), optionally a step height (Δf), and optionally a step width (σ).

29. The method of claim 27, wherein said skin-type values are a function of a step depth (d₀) and/or a step height (Δf), and/or a step width (σ).

30. The method of claim 18, wherein said skin type values define a location of a transition between cutis and subcutis, and/or comprise a thickness of cutis and/or subcutis.

31. (canceled)

32. The method of claim 18, wherein said NMR parameters comprise one or more parameters selected from the group consisting of: relaxation time T2 (true), relaxation time T2 (effective), relaxation time T1 (true), relaxation time T1 (effective), self-diffusion coefficient D, signal amplitude, spin modes, pulse sequence CPMG, dipolar encoded longitudinal magnetization, multiquantum build-up, multiquantum decay, diffusion coefficients, chemical shift resolved spectra, component amplitudes in chemical shift resolved spectra, and/or a mathematical function thereof.

33. The method of claim 18, wherein at least a plurality of records comprise one or more of the following
NMR data characterizing the skin of a region of a reference subject; skin-type values calculated from said NMR data, Glogau scale value, and Fitzpatrick scale value.

34. The method of claim 18, wherein at least a plurality of records comprise one or more of the following
a step depth (d₀), a step height (Δf), a step width (σ), a location of a transition between cutis and subcutis, a thickness of cutis, a thickness of subcutis, skin thickness, skin hardness, skin water content, skin color, freckles, and scaling

35. The method of claim 18, wherein at least a plurality of records comprise one or more of the following NMR parameters: the relaxation time T2 (true), relaxation time T2 (effective), relaxation time T1 (true), relaxation time T1 (effective), self-diffusion coefficient D, signal amplitude, spin modes, pulse sequence CPMG, dipolar encoded longitudinal magnetization, multiquantum build-up, multiquantum decay, diffusion coefficients, chemical shift resolved spectra, component amplitudes in chemical shift resolved spectra, and/or a mathematical function thereof.

36. The method of claim 18, wherein said skin type records further comprise one or more parameters selected from the group consisting of reference subject identifier, reference subject age, reference subject gender, reference subject ethnicity, and reference subject skin type sample location.

37. The method according of claim 18, wherein said receiving one or NMR parameters determined from said region of skin comprises receiving one or more skin NMR parameters selected from the group consisting of the relaxation time T2 (true), relaxation time T2 (effective), relaxation time T1 (true), relaxation time T1 (effective), self-diffusion coefficient D, signal amplitude, spin modes, pulse sequence CPMG, dipolar encoded longitudinal magnetization, multiquantum build-up, multiquantum decay, diffusion coefficients, chemical shift resolved spectra, component amplitudes in chemical shift resolved spectra, and/or a mathematical function thereof.

38. The method of claim 18, wherein said receiving further comprises receiving one or more parameters selected from the group consisting of a treatment subject identifier, a treatment subject age, a treatment subject gender, a treatment subject ethnicity, and a treatment subject skin type sample location.

39. The method of claim 18, wherein said outputting comprises outputting comprises storing to a computer readable medium selected from the group consisting of a magnetic medium, an optical medium, and a flash memory.

40. The method of claim 39, wherein said outputting comprises outputting to a patient record.

41. The method of claim 18, wherein said providing a skin type database containing skin type records from a plurality of subjects comprises accessing a remote skin type database.

42. A machine-accessible medium that provides instructions that, if executed by a machine, will cause the machine to perform operations comprising:
receiving one or more nuclear magnetic resonance (NMR) parameters and/or skin-type values calculated from said NMR parameters and/or calculating skin-type values from said NMR parameters wherein said NMR parameters are from NMR measurements from skin at one or more locations on a subject and said skin-type values are indicative of a skin characteristic; and
determining and outputting to a display or tangible medium, a treatment plan optimized for a skin type characterization determined from said skin-type values.

43. The medium of claim 42, wherein said skin-type values are indicative of a Glogau and/or a Fitzpatrick scale value.

44. The medium of claim 42, wherein said calculating skin-type values comprises querying a skin-type skin type database to identify the skin type values characterized by said NMR parameters.

45. The medium of claim 42, wherein said NMR parameters are determined using a portable NMR device.

46-51. (canceled)

52. The medium of claim 42, wherein said receiving comprises calculating or receiving already calculated a step depth (d₀), optionally a step height (Δf), and optionally a step width (σ).

53. The medium of claim 42, wherein said skin-type values are a function of a step depth (d₀) and/or a step height (Δf), and/or a step width (σ).

54. The medium of claim 42, wherein said skin type values define a location of a transition between cutis and subcutis and/or comprise a thickness of cutis and/or subcutis.

55-56. (canceled)

57. The medium of claim 42, wherein said operations further comprise outputting treatment operation parameters to a device selected from the group consisting of laser, a radiofrequency device, a plasma generator, a pulsed light generator.

58-66. (canceled)

67. A system comprising a computer processor configured to:
receive NMR data obtained from a subject's skin and to calculate a skin-type value from said NMR data and/or to query a database storing a library of NMR skin type characterizations to return one or more skin-type values for said skin, wherein said skin-type values are indicative of a skin characteristic.

68. The system of claim 67, wherein said skin-type values are indicative of a Glogau and/or a Fitzpatrick scale value.

69. The system of claim 67, further wherein said processor or a second processor is configured to generate a treatment plan optimized for a patient skin type characterized by a said NMR data.

70. The system of claim 67, wherein said system further comprises an NMR measuring device.

71. The system of claim 67, wherein said system further comprises a portable NMR device.

72-83. (canceled)

84. A method of treating a region of interest of the skin of a subject, said method comprising:
identifying a region of interest of the skin of a subject to be treated;
making one or more NMR measurements of said region to obtain NMR parameters characterizing the skin region;
calculating one or more skin-type values and/or querying an NMR skin type database with the NMR data and/or skin-type values to identify the skin type characterized by said NMR data;
calculating and outputting to a display or tangible medium, a treatment plan optimized for the skin type characterization returned from said query; and
treating said subject in accordance with said treatment plan.

85. The method of claim 84, wherein said calculating comprises analyzing a signal comprising NMR signal amplitude as a function of skin depth at a location skin location to determine a step depth (d₀), optionally a step height (Δf), and optionally a step width (σ) and/or calculating comprises calculating a skin-type value that is a function of a step depth (d₀) and/or a step height (Δf), and/or a step width (σ).

86. (canceled)

87. The method of claim 86, wherein said skin type value defines a location of a transition between cutis and subcutis, and/or comprises a thickness of cutis and/or subcutis.

88. (canceled)

89. The method of claim 84, wherein said treating comprises outputting operation parameters to a device selected from the group consisting of laser, a radiofrequency device, a plasma generator, a pulsed light generator, and/or selecting a pharmaceutical and/or cosmetic regimen.

90-98. (canceled)

99. A method of generating a skin type database used for treating a region of skin of interest, the method comprising:
making one or more NMR measurements of a skin region of interest in a reference subject; and
storing a plurality of parameters obtained from said NMR measurement(s) and/or one or more skin-type values derived from said NMR measurement(s), in a computer readable medium to form a skin type database.

100-109. (canceled)
